(19) United States
(12) Patent Application Publication
(10) Pub. No.: US 2022/0387368 A1
(43) Pub. Date: Dec. 8, 2022

(54) AN INHIBITOR OF AN IRON-CONTAINING HYDROGENASE OF A METHANOGENIC ARCHAEON FOR USE IN THE TREATMENT OR PREVENTION OF A PARKINSONIAN CONDITION

(71) Applicant: UNIVERSITE DU LUXEMBOURG, ESCH-SUR-ALZETTE (LU)

(72) Inventors: Jean-Pierre Trezzi, Rodange (LU); Paul Wilmes, Bettembourg (LU)

(21) Appl. No.: 17/776,001

(22) PCT Filed: Nov. 12, 2020
(86) PCT No.: PCT/EP2020/081855

§ 371 Date: May 11, 2022

(30) Foreign Application Priority Data
Nov. 12, 2019 (LU) LU101477

(51) Int. Cl. A61K 31/22; A61K 31/366; A61K 31/4045; A61P 25/16

(52) U.S. Cl. CPC A61K 31/22; A61K 31/366; A61K 31/4045; A61P 25/16

(57) ABSTRACT

The present invention relates to an inhibitor of an iron-containing hydrogenase of a methanogenic archaeon for use in the treatment or prevention of a parkinsonian condition, wherein said archaeon is in the gut of a subject and is characterized by the production of 2-hydroxypyridine. In addition, the present invention relates to a pharmaceutical composition comprising such an inhibitor and a kit comprising such an inhibitor. Further, the present invention relates to a method of screening for an inhibitor for use according to the present invention and a method for determining whether or not a subject is susceptible to a treatment with an inhibitor for use according to the present invention.

The work leading to this invention received support from the Luxembourg Fonds National de la Recherche (FNR) under grant No. CORE 11333923 and the Michael J. Fox Foundation under grant No. 14701.

### TECHNICAL FIELD

The present invention relates to an inhibitor of an iron-containing hydrogenase of a methanogenic archaeon for use in the treatment or prevention of a parkinsonian condition, wherein said archaeon is in the gut of a subject and is characterized by the production of 2-hydroxypyridine. In addition, the present invention relates to a pharmaceutical composition comprising such an inhibitor and a kit comprising such an inhibitor. Further, the present invention relates to a method of screening for an inhibitor for use according to the present invention and a method for determining whether or not a subject is susceptible to a treatment with an inhibitor for use according to the present invention.

### BACKGROUND OF THE INVENTION

Neurological disorders have become the leading cause of disability in the world, and Parkinson's disease (PD) is one of the most significant medical and social burdens of our time. With the aging population, the number of people suffering from PD is expected to double from 6.9 million in 2015 to 14.2 million in 2040 (Dorsey et al., 2018). PD is a complex, progressive, and widely systemic neurodegenerative disease characterized by a number of motor and non-motor symptoms. PD has a long (up to 20-30 years of) prodromal period, during which several non-motor features can develop, including impairment in olfaction, constipation and rapid eye movement sleep behaviour disorder (RBD) (Chaudhuri et al., 2009; Schrag et al., 2015).

Previous investigations of biomarkers in biological fluids (blood and cerebrospinal fluid) have neither yielded diagnostic nor progression markers for early detection (Simonsen et al., 2016; Mollenhauer Zimmermann et al., 2016). The underlying pathogenesis of PD is still elusive, and biomarkers are important for upcoming clinical trials with putative disease-modifying agents (Olanow et al., 2010).

The defining pathology in PD is the death of dopaminergic neurons in a part of the midbrain called the substantia nigra. Another hallmark of PD is an accumulation of neuronal inclusions, known as Lewy bodies, in various parts of the brain and body (such as the substantia nigra, cerebral cortex, dorsal nucleus of the vagus nerve, sympathetic ganglia, and the myenteric plexus of the intestines). These Lewy bodies contain misfolded alpha-synuclein, ubiquitin, complement proteins, and cytoplasmic structural proteins. The propagation of α-synuclein aggregations in the form of intracellular Lewy body inclusions in PD has been shown to start peripherally in the enteric nervous system (ENS; Braak et al., 2006) and the olfactory bulb (Braak et al., 2006; Braak et al., 2003) before affecting the central nervous system (CNS) resulting in a staged topographic ascending distribution pattern of intracerebral lesions.

In this context, emerging studies on the subject of PD and the microbiota have begun to focus on the potential mechanisms by which the microbiota contribute to the formation of alpha-synuclein pathogenic species in the ENS and CNS. Within the gut microbiome, PD-specific signatures have recently been reported (Scheperjans et al., 2015a; Scheperjans et al., 2015b). However, at present, it remains unclear whether the differences in the gut microbiome of PD patients are a consequence of the disease or causally related to the presence of causative microbial agents, such as toxin-producing organisms.

Despite the immediate research interest and the relatively easy accessibility (once standard operating procedures are established), not a single published study on the gut microbiome exists in the context of early and longitudinally followed PD, and at-risk subjects (RBD). The only previous studies of the gut microbiome of PD patients have used a phylogenetic marker sequencing approach (16S rRNA gene amplicon sequencing), which allows for the taxonomic identification and relative quantification of microorganisms, thereby not allowing a realistic assessment of the vitality, functional potential or functional activity of these taxa and therefore not conveying important disease-relevant information (Scheperjans et al., 2015b; Keshavarzian et al., 2015).

Independently of the microbiome, recent studies (Horvath et al., 2012) have shown that the ring-fused 2-pyridone molecule (FN075) inhibits fibrillation of amyloidogenic Curli protein CsgA and interestingly stimulates alpha-synuclein amyloid fiber formation. Thereby, the Curlicide/Pilicide with a reactive ring-fused 2-pyridone seems to have an impact on alpha-synuclein aggregation/fibrillation, as observed in Parkinson's disease. Interestingly, in vivo approaches (Chermenina et al., 2015; Cairns et al., 2018) that delivered 2-pyridone fibrillization modulators to both mice and non-human primate were developed and resulted in symptoms mimicking early stages of Parkinson's disease. However, no correlation between the ring-fused 2-pyridone and the microbiome has been shown.

The molecule 2-HP is a known co-factor of archaeal iron-containing hydrogenases (Vogt et al., 2008) and a degradation product of the abundant pesticide chlorpyrifos. Harishankar et al. (2013) shows that the chlorpyrifos is the main source of the metabolite 3,5,6-trichloro-2-pyridinol (TCP), which—in turn—may be transformed into 2-HP, probably via dechlorination by bacteria, including members of the gut microbiome, such as Lactobacillus spp.

There is a need for new therapeutic interventions to prevent and/or treat diseases, states or syndromes of a parkinsonian condition. The present invention addresses all these needs. Accordingly, the technical problem underlying the present application is to comply with these needs.

### SUMMARY OF THE INVENTION

The inventors found 2-hydroxypyridine (2-HP or 2HP in the following) as a key molecule involved in the pathogenesis and progression of a parkinsonian condition, for example, Parkinson's disease, Parkinsonism, Parkinson-plus syndrome, early-stage idiopathic Parkinson's disease (PD) and prodromal Rapid eye movement (REM) sleep behaviour disorder (RBD). Providers of 2-hydroxypyridine can be found in the microbiome of patients of PD. If in contact with alpha-synuclein, 2-hydroxypyridine promotes aggregation, which is known to be associated with Parkinson's disease. This reveals 2-HP, an archaeal cofactor and pesticide degradation product, not only as being a key molecule involved in the pathogenesis and progression of idiopathic PD, but provides also a mechanistic insight of the involvement of 2-HP as a driver of Parkinson's disease pathology. Therefore, any inhibitor, which targets the production of 2-hydroxypyridine, mainly in the gut of a subject, is therefore relevant for use in the treatment or prevention of a parkinsonian condition.

The present invention relates to an inhibitor of an iron-containing hydrogenase of a methanogenic archaeon for use in the treatment or prevention of a parkinsonian condition, wherein said archaeon is in the gut of a subject and is characterized by the production of 2-hydroxypyridine. The present invention also relates to an inhibitor of an iron-containing hydrogenase of a methanogenic archaeon for use in the treatment or prevention of a parkinsonian condition.

In a preferred embodiment, the present invention is directed to an inhibitor of an iron-containing hydrogenase of a methanogenic archaeon for use in the treatment or prevention of a parkinsonian condition, wherein said archaeon is comprised in the gut of a subject and is characterized by the production of 2-hydroxypyridine. In an also preferred embodiment, the present invention is directed to an inhibitor of an iron-containing hydrogenase of a methanogenic archaeon for use in the treatment or prevention of a parkinsonian condition.

In a preferred embodiment of the inhibitor for use according to the present invention, the inhibitor for use prevents or reduces the production of 2-hydroxypyridine. More preferably, the inhibitor for use prevents or reduces the production of 2-hydroxypyridine by the methanogenic archaeon by blocking the synthesis pathway of 2-hydroxypyridine. In a more preferred embodiment, the inhibitor for use prevents or reduces the production of 2-hydroxypyridine by the methanogenic archaeon by targeting the methanogenic archaeon. In an also preferred embodiment of the inhibitor for use according to the present invention, the inhibitor for use prevents or reduces the production of 2-hydroxypyridine by preventing the degradation of a precursor of 2-hydroxypridine, more preferably wherein the precursor of 2-hydroxypridine is derived from a pesticide, most preferably wherein the pesticide is chlorpyrifos.

In an also preferred embodiment of the inhibitor for use according to the present invention, the inhibitor is selected from the group consisting of statins, preferably atorvastatin, fluvastatin, lovastatin, pitavastatin, pravastatin, rosuvastatin or simvastatin, and antibiotics, preferably rifaximin or neomycin.

In a further preferred embodiment of the inhibitor for use according to the present invention, the 2-hydroxypyridine comprises the compound of general formula I,

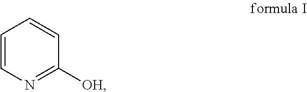

an isomer of said 2-hydroxypyridine or a mixture of isomers thereof, a derivative of said 2-hydroxypyridine or a mixture of derivatives thereof, a pharmaceutically acceptable salt of said 2-hydroxypyridine or a mixture of pharmaceutically acceptable salts thereof; or a complex of said 2-hydroxypyridine or a mixture of complexes thereof.

In an also preferred embodiment of the inhibitor for use according to the present invention, the inhibitor prevents or reduces the production of 2-hydroxypyridine in the gut of said subject.

In a further preferred embodiment of the inhibitor for use according to the present invention, the inhibitor is a small molecule, preferably a small molecule having a molecular weight of less than 2000 daltons; or a biologic drug, preferably a peptide, a protein, an antibody or an antibody fragment.

In an also preferred embodiment of the inhibitor for use according to the present invention, the inhibitor is for use in the treatment or prevention of a parkinsonian condition in co-treatment with other compounds, which are preferably selected from the group consisting of carbidopa-levodopa, dopamine agonists, MAO B inhibitors, catechol O-methyltransferase (COMT) inhibitors, anticholinergics, amantadine and antibiotics.

In a further preferred embodiment of the inhibitor for use according to the present invention, the subject is administered a therapeutically effective amount of the inhibitor, preferably at least 0.1 mg.

In an also preferred embodiment of the inhibitor for use according to the present invention, the subject is administered a dosage of the inhibitor in the range of 0.1 to 1.0 mg per day.

In a further preferred embodiment of the inhibitor for use according to the present invention, the parkinsonian condition is selected from the group consisting of Parkinson's disease, Parkinsonism, Rapid Eye Movement Sleep Behavior Disorder and Parkinson-plus syndrome.

The present invention also relates to a kit comprising the inhibitor for use according to the present invention as described herein.

In a preferred embodiment of the kit according to the present invention, the kit further comprises a pharmaceutically acceptable carrier and/or an antiparkinsonian agent, preferably said antiparkinsonian agent is one or more of the following: L-DOPA, deprenyl, apomorphine, an anticholinergic agent, further preferably said anticholinergic agent is selected from the group consisting of benzhexol and orphenadrine.

Further, the present invention also relates to a pharmaceutical composition comprising the inhibitor for use according to the present invention.

The present invention also relates to a method of screening for an inhibitor for use according to present invention and as described elsewhere herein, wherein the method comprises (i) contacting a compound of interest with an iron-containing hydrogenase of a methanogenic archaeon, (ii) measuring inhibitor function of the compound of interest for the iron-containing hydrogenase of the methanogenic archaeon, wherein an increase of inhibition of said iron-containing hydrogenase compared to the iron-containing hydrogenase before contacting of step (i) indicates that the compound of interest serves as an inhibitor for the iron-containing hydrogenase, or wherein a decrease or maintenance of inhibition of said iron-containing hydrogenase compared to the iron-containing hydrogenase before contacting of step (i) indicates that the compound of interest does not serve as an inhibitor for the iron-containing hydrogenase.

The present invention also relates to a method for determining whether or not a subject is susceptible to a treatment with an inhibitor for use according to the present invention, comprising determining the concentration of 2-hydroxypyridine in a sample obtained from the gut of said subject, compared to a control sample of the same subject.

### BRIEF DESCRIPTION OF THE DRAWINGS

FIG. 1 shows significant changes (p=0.0007, FDR=0.006) in the abundance of 2-HP. The levels of this metabolite are significantly higher in PD and RBD compared to healthy controls, but not significantly altered between PD and RBD. Semi-quantitative levels of 2-HP have been determined by GC-MS analysis from faecal samples derived from 45 PD patients, 30 iRBD subjects as well as 50 healthy control subjects.

FIG. 2 shows significant changes (p=0.00005, FDR=0.005) in the abundance of beta-glutamic acid. The levels of this metabolite are significantly higher in RBD compared to healthy controls, but not significantly altered between PD and controls. Semi-quantitative levels of beta-glutamic acid have been determined by GC-MS analysis from faecal samples derived from 45 PD patients, 30 iRBD subjects as well as 50 healthy control subjects.

FIG. 3 shows toxicity assays of 2-HP in HiTox strains of yeast expressing human alpha-synuclein. 2-HP is selectively toxic at doses of 1 mM to transgenic alpha-synuclein expressing yeast. FIG. 3A shows that in presence of raffinose, alpha-synuclein is not induced. A toxic dose-response can be observed. FIG. 3B shows that in presence of galactose, alpha-synuclein is induced, leading to growth defects. A toxic dose-response can be observed in the HiTox strains, but not in the control strain. FIG. 3C shows the growth curve in presence and absence of 2-HP. At higher concentration of 2-HP, the lag time in the control is prolonged. However, the yield is unaffected.

FIG. 4 shows quantitative alpha-synuclein aggregation assays in HiTox strains of yeast expressing human alpha-synuclein after treatment with 2-HP. The aggregation of alpha-synuclein is the key molecular hallmark of PD.

FIG. 5 shows the toxicity of 2-HP in enteric neurons. LD₅₀ seems to be located around 3 mM. In addition to the yeast model, the acute toxicity and alpha-synuclein induced aggregation of 2-HP in iPS cells differentiated to a proxy of the cells present in the enteric nervous system have been assessed. At 3 mM, the inventors have observed significant changes in the toxicity of 2-HP on these cells.

FIG. 6 shows semi-quantitative alpha-synuclein aggregation assays in enteric neurons after treatment with 2-HP. The following abbreviations have the meaning given in brackets: α-Syn (alpha-synuclein), α-Syn-F (fibrillary alpha-synuclein, i.e. alpha-synuclein aggregations), Hoechst (DNA staining), TUJ1 (general neuronal marker), TH (tyrosine hydroxylase) and CC3 (cleaved caspase 3 staining, i.e. cell death marker, showing toxicity).

FIG. 7 shows a schematically overview/procedure of the mouse model for Parkinson's disease expressing human alpha-synuclein.

FIG. 8 shows the effects of 2-hydroxypyridine on IL-1b mRNA expression in murine BMDM model.

FIG. 9 shows the relative abundance of genus Methanobrevibacter in two cohorts.

FIG. 10 shows the concentrations of 2-hydroxypyridine in cell pellets from archaeal cultures (targeted GC/MS).

FIG. 11 shows an adhesive removal test in mice, 15 days post 2-HP injection. The following abbreviations have the meaning given in brackets: WT (wildtype mice), TG (transgenic mice overexpressing human alpha-synuclein). n=5 for each group/concentration.

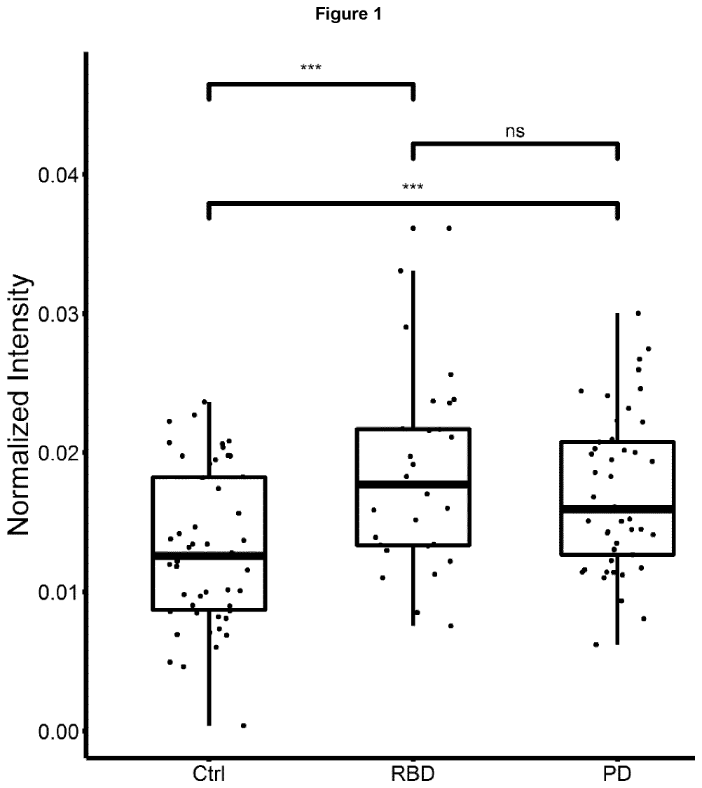

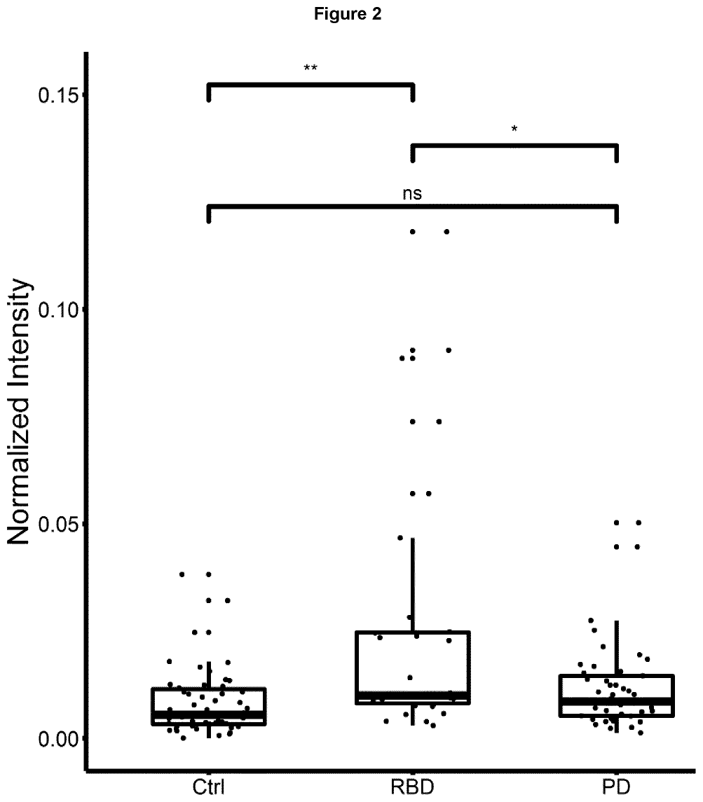

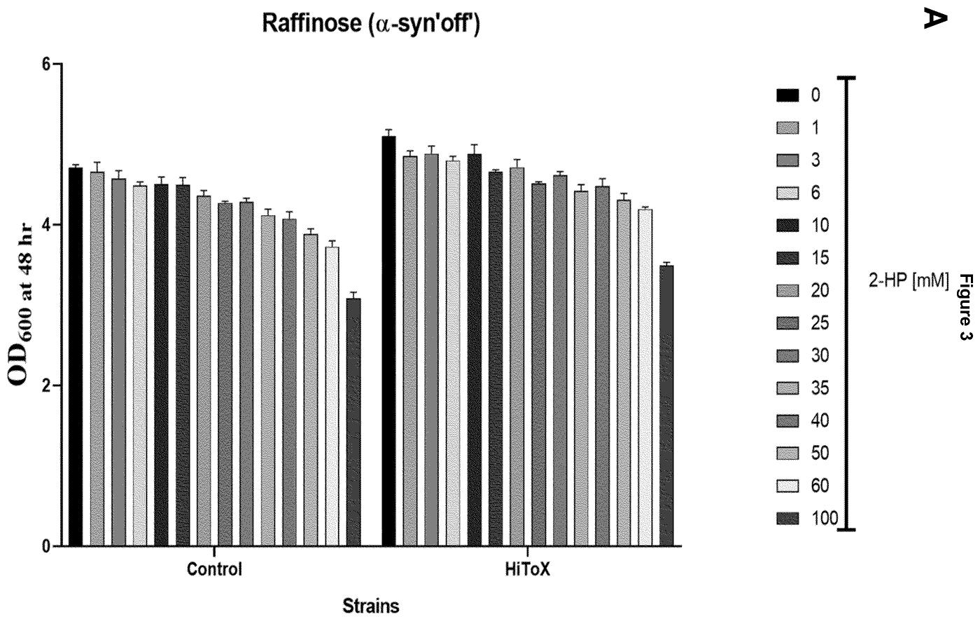

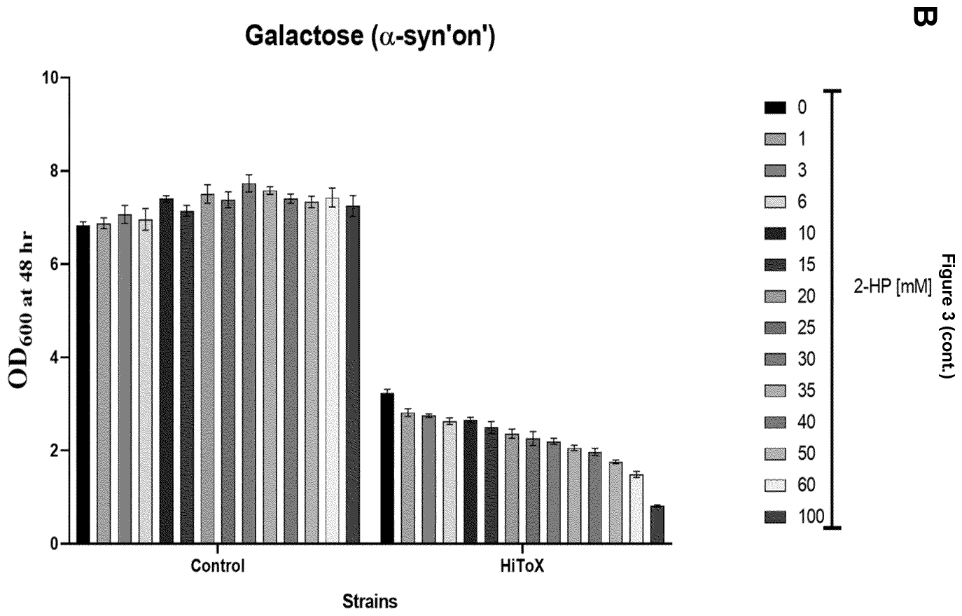

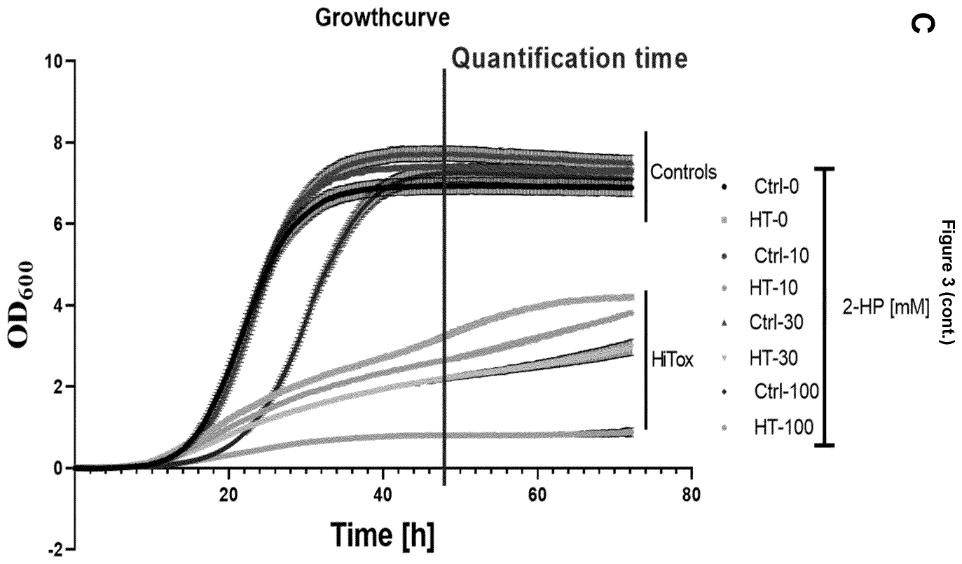

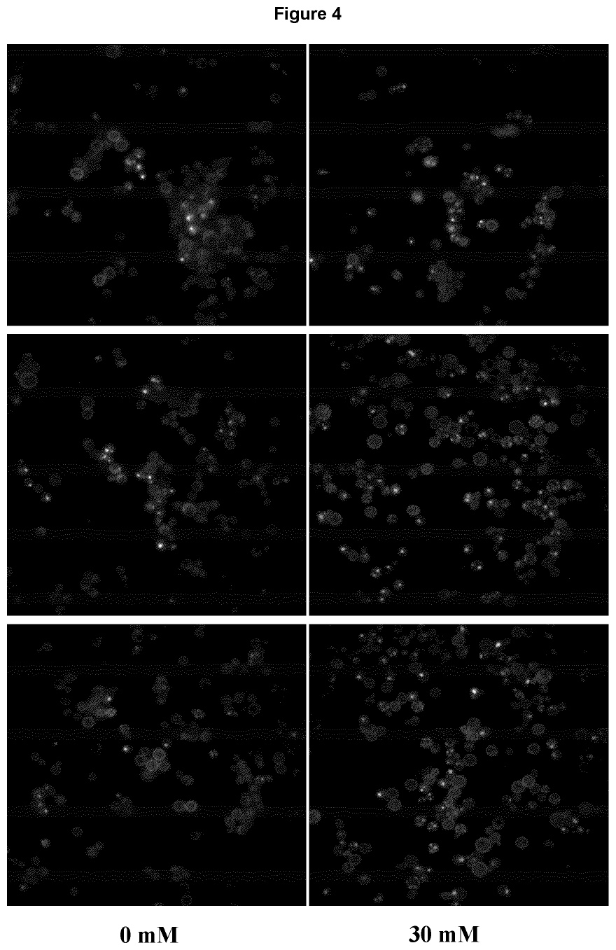

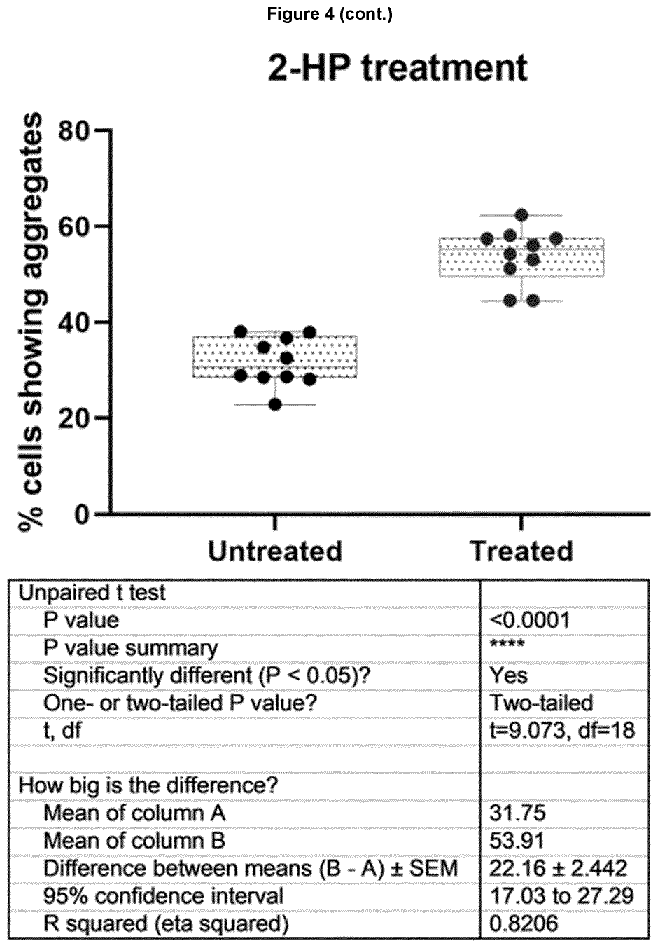

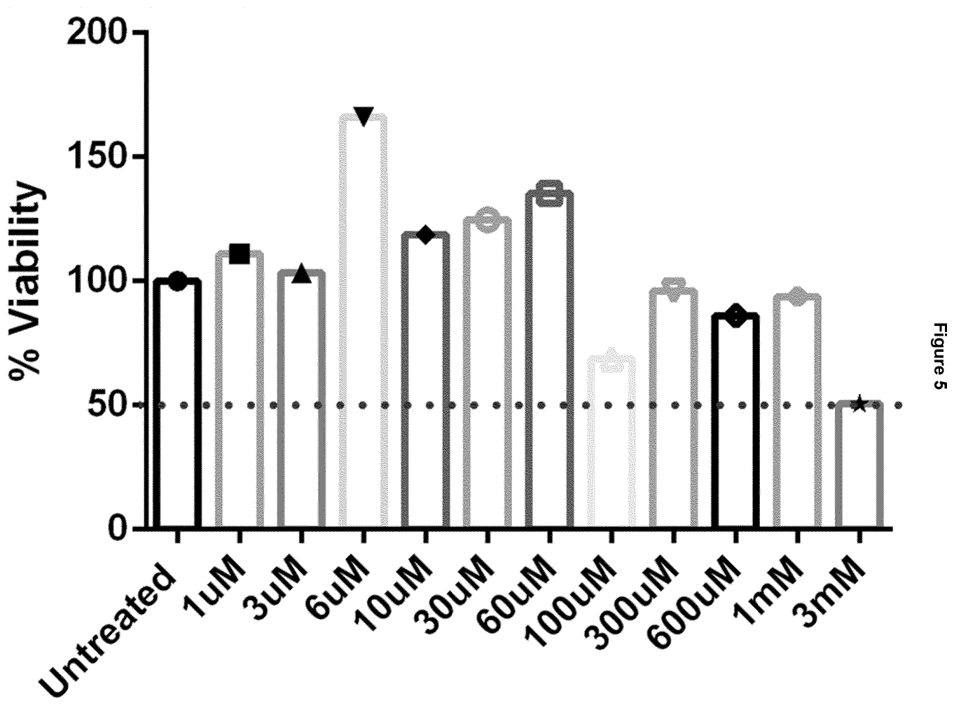

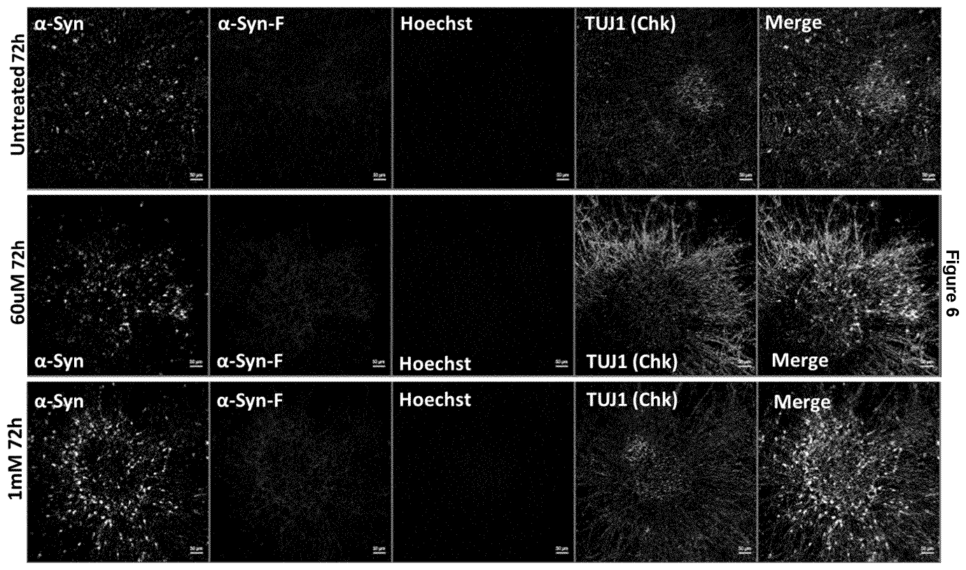

### DETAILED DESCRIPTION OF THE INVENTION

The following detailed description refers to the accompanying Examples and Figures that show, by way of illustration, specific details and embodiments, in which the invention may be practised. These embodiments are described in sufficient detail to enable those skilled in the art to practice the invention. Other embodiments may be utilized such that structural, logical, and eclectic changes may be made without departing from the scope of the invention. Various aspects of the present invention described herein are not necessarily mutually exclusive, as aspects of the present invention can be combined with one or more other aspects to form new embodiments of the present invention.

The present invention relates to an inhibitor of an iron-containing hydrogenase of a methanogenic archaeon for use in the treatment or prevention of a parkinsonian condition, wherein said archaeon is in the gut of a subject and is characterized by the production of 2-hydroxypyridine. The present invention also relates to an inhibitor of an iron-containing hydrogenase of a methanogenic archaeon for use in the treatment or prevention of a parkinsonian condition. The present invention also relates to an inhibitor of an iron-containing hydrogenase of a methanogenic archaeon for use in the treatment or prevention of a parkinsonian condition, wherein said archaeon is comprised in the gut of a subject and is characterized by the production of 2-hydroxypyridine.

The term “inhibitor” as used herein and as used in the context of the present invention means an inhibitor that inhibits or delays enzyme reactions. In principle, depending on the type of inhibitor, different forms can be distinguished when inhibiting enzyme reactions, for example, competitive inhibitions or inhibitors, wherein the inhibitor competes with the substrate (the substance to be implemented) or allosteric inhibition or inhibitors, wherein the inhibitor changes the molecular structure of the enzyme, so that the substrate can no longer be bound and therefore cannot be converted. A special form of competitive inhibition is product inhibition. In this form of inhibition, the product itself regulates the enzymatic process by inhibiting an enzyme. According to the present invention, the inhibitor has the ability to inhibit the reaction catalyzed by a hydrogenase, specifically an iron-containing hydrogenase, and most specifically an iron-containing hydrogenase of a methanogenic archaeon. However, of course, the present invention also comprises an inhibitor of one or more than one iron-containing hydrogenase(s) of a methanogenic archaeon for use in the treatment or prevention of a parkinsonian condition, wherein said archaeon is in the gut of a subject and is characterized by the production of 2-hydroxypyridine. Further, the present invention also comprises an inhibitor of one or more than one iron-containing hydrogenase(s) of one or more than one methanogenic archaeon/archaea for use in the treatment or prevention of a parkinsonian condition, wherein said archaeon/archaea is/are in the gut of a subject and is/are characterized by the production of 2-hydroxypyridine.

As used herein, the term “iron-containing hydrogenase” means an enzyme that catalyzes the reversible reduction of methenyltetrahydromethanopterin (methenyl-H₄MPT⁺) with H₂ to methenyl-H₄MPT, a reaction involved in methanogenesis from H₂ and CO₂ in many methanogenic archaea. The enzyme harbors an iron-containing cofactor, in which a low-spin iron is complexed by a pyridone, two CO and a cysteine sulfur. An iron-containing hydrogenase is thus similar to [NiFe]- and [FeFe]-hydrogenases, in which a low-spin iron carbonyl complex, albeit in a dinuclear metal center, is also involved in H₂ activation. Thus, like the [NiFe]- and [FeFe]-hydrogenases, the iron-containing hydrogenase catalyzes an active exchange of H₂ with protons of water; however, this activity is dependent on the presence of the hydride-accepting methenyl-H₄MPT⁺. The iron-containing hydrogenase may also be called [Fe]-hydrogenase or [Fe]-hydrogenase (iron-sulfur-cluster-freehydrogenase). In one preferred embodiment of the present invention, the iron-containing hydrogenase may be 5,10-methenyltetrahydromethanopterin hydrogenase.

In the context of the present invention, the term “methanogenic archaeon” (singular) or “methanogenic archaea” (plural) refers to a phylogenetically diverse group of strictly anaerobic Euryarchaeota with an energy metabolism that is restricted to the formation of methane from CO₂. Most methanogenic archaea can reduce CO₂ with H₂ to methane, and it is generally assumed that the reactions and mechanisms of energy conservation that are involved are largely the same in all methanogens. The term “methanogenic archaeon” (singular) or “methanogenic archaea” (plural), as used within the context of the present invention also comprises hydrogenotropic, methanogenic archaeon or hydrogenotropic, methanogenic archaea. Examples of strains of methanogenic archaea include Methanobacterium, Methanobrevibacter, Methanococcus and Methanothermobacter. In one preferred embodiment of the present invention, the methanogenic archaeon is from Methanobrevibacter, more preferably Methanobrevibacter smithii and Methanosphaera stadtmanae.

As used herein, the term “treating” and “treatment” refers to administering to a subject a therapeutically effective amount of an inhibitor for use according to the present invention or a pharmaceutical composition according to the present invention. A “therapeutically effective amount” may refer to an amount of the inhibitor for use according to the present invention or the pharmaceutical composition according to the present invention, which is sufficient to treat or ameliorate a disease or disorder, to delay the onset of a disease or to provide any therapeutical benefit in the treatment or management of a disease.

As used herein, the term “prophylaxis” or “prevention” refers to the use of an agent for delaying, stopping or hindering the onset of a disease, disorder, syndrome or condition. A “prophylactically effective amount” defines an amount of the inhibitor for use according to the present invention or of the pharmaceutical composition according to the present invention sufficient to prevent the onset or recurrence of a disease.

As used herein, the term “parkinsonian condition” refers to a clinical/pathological condition (e.g., clinical situation), disease, state (e.g., pathological state) or syndrome comprising the following symptoms: Tremor, bradykinesia, rigidity, and postural instability. Preferably, said parkinsonian condition is selected from the group consisting of: Parkinson's disease, Parkinsonism, Rapid Eye Movement Sleep Behavior Disorder (RBD) and Parkinson-plus syndrome.

As used herein, the term “parkinsonism” refers to a clinical/pathological condition (e.g., clinical situation) characterized by (e.g., consisting of) the following symptoms: Tremor, bradykinesia, rigidity and postural instability. Preferably, said Parkinsonism comprises one or more of the following conditions: Drug induced parkinsonism, toxin induced parkinsonism and secondary parkinsonism. Preferably, said drug induced parkinsonism is selected from the group consisting of parkinsonism induced by one or more of the following: Neuroleptics, antipsychotics, lithium, metoclopramide, MDMA, tetrabenazine. Preferably, said secondary parkinsonism is selected from the group consisting of malignant neuroleptic syndrome; drug-induced secondary parkinsonism, secondary parkinsonism due to other external agents; post-encephalitic parkinsonism; vascular parkinsonism; and syphilitic parkinsonism.

As used herein, the term “Parkinson's disease” refers to a clinical/pathological condition (e.g., clinical situation) characterized by (e.g., consisting of) the following symptoms: Tremor, bradykinesia, rigidity, postural instability and cognitive impairment. Preferably, said Parkinson's disease comprises one or more of the following: Parkinsonism, Hemiparkinsonism; Paralysis agitans; idiopathic Parkinson's disease; primary Parkinson's disease, Parkinson's disease dementia.

As used herein, the term “Parkinson-plus syndrome” refers to a clinical/pathological condition (e.g., clinical situation) characterized by the following symptoms: Tremor, bradykinesia, rigidity, postural instability with additional symptoms that distinguish it from Parkinson's disease. Preferably, said Parkinson-plus syndrome is selected from the group consisting of atypical parkinsonism; multiple system atrophy (MSA); progressive supranuclear palsy (PSP); corticobasal degeneration (CBD); dementia with Lewy bodies (DLB); Pick's disease; and olivopontocerebellar atrophy.

These conditions usually lead to the death of dopaminergic neurons of the subject. As used herein, the term “dopaminergic neuron” refers to a neuron that releases dopamine from its synapses. Non-limiting examples of dopaminergic neurons include dopaminergic neurons present in the ventral tegmental area of the midbrain, substantia nigra pars compacta, and arcuate nucleus of the hypothalamus. Dopaminergic neurons may also be tyrosine hydroxylase positive (TH⁺) dopaminergic neurons (e.g., tyrosine hydroxylase is the tyrosine 3-monooxygenase having UniProtKB Accession Number: P07101). Dopaminergic neurons may be obtainable from the mammalian midbrain region, preferably said midbrain region is a substantia nigra, further preferably said midbrain region is pars compacta portion of substantia nigra; most preferably said dopaminergic neurons are obtainable from said midbrain region of a subject (e.g., human) diagnosed with a parkinsonian condition selected from the group consisting of Parkinson's disease, Parkinsonism, RBD and Parkinson-plus syndrome.

As used herein, the term “Rapid Eye Movement (REM) Sleep Behavior Disorder (RBD)” refers to a sleep disorder in which one physically acts out vivid, often unpleasant dreams with vocal sounds and sudden, often violent arm and leg movements during REM sleep—sometimes called dream-enacting behavior. Symptoms of REM sleep behavior disorder may include: Movement, such as kicking, punching, arm flailing or jumping from bed, in response to action-filled or violent dreams, such as being chased or defending yourself from an attack; noises, such as talking, laughing, shouting, emotional outcries or even cursing; and being able to recall the dream if you awaken during the episode. Thus, the major feature of RBD is the loss of muscle atonia (i.e., the loss of paralysis) during otherwise intact REM sleep (during which paralysis is not only normal, but necessary). REM sleep is the stage of sleep in which most vivid dreaming occurs. The loss of motor inhibition leads to a wide spectrum of behavioral release during sleep. Rapid Eye Movement (REM) Sleep Behavior Disorder (RBD) is a very strong predictor or indicator of progression to a synucleinopathy, including Parkinson's disease or dementia with Lewy bodies.

The term “subject”, when used herein, can be a vertebrate such as a mammal. Mammals include, but are not limited to, farm animals, sport animals, pets, primates, mice and rats. Preferably, a mammal is a human, dog, cat, cow, pig, mouse, rat, etc. Human beings are preferred. The “subject”, which may be treated with one or more inhibitors for use according to the present invention or pharmaceutical compositions as described herein, can be a vertebrate, preferably a vertebrate that has an adaptive immune system. In the context of the present invention, the term “subject” can mean an individual in need of a treatment and/or prophylaxis of a parkinsonian condition. The subject can also be a patient suffering from a parkinsonian condition, like Parkinson's disease, Parkinsonism, Rapid Eye Movement Sleep Behavior Disorder (RBD) and Parkinson-plus syndrome.

In the context of the present invention, the term “2-hydroxypyridine” means a compound, being of the general formula as given below:

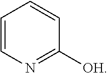

However, the term “2-hydroxypyridine” as used in the context of the present invention also includes an isomer of said 2-hydroxy-pyridine compound or a mixture of isomers thereof; a tautomer of said 2-hydroxypyridine compound or a mixture of tautomers thereof, a derivative of said 2-hydroxypyridine compound or a mixture of derivatives thereof, a pharmaceutically acceptable salt of said 2-hydroxypyridine compound or a mixture of pharmaceutically acceptable salts thereof; or a complex of said 2-hydroxypyridine compound or a mixture of complexes thereof. The term “2-hydroxypyridine” as used within the context of the present invention can be used synonymously to the terms 1,2-dihydro-2-oxopyridine, 1H-2-pyridone, 1H-pyridin-2-one, 2(1H)-PYRIDONE, 2-HYDROXYPYRIDINE, 2-oxopyridine, 2-PYRIDINOL, 2-pyridinone, 2-PYRIDOL, 2-PYRIDON, 2-PYRIDONE, alpha-hydroxypyridine, A-PYRIDONE, AURORA KA-3075, PYRIDIN-2-OL, TIMTEC-BB SBB004392, 1-hydroxy-2-pyridine, 2(1H)-pyridinone, alpha-pyridone and pyridone-2.

As used herein, the term “isomer” refers to a compound having the same molecular formula, but differing in the nature or order of bonding of its atoms or in the spatial arrangement of its atoms. Isomers that differ in the arrangement of their atoms in space are referred to as “stereoisomers”. “Stereoisomer” refers to a compound that exists in a different stereoisomeric form if one or more asymmetric centers or asymmetrically substituted double bonds are present and thus can be produced as individual stereoisomers or mixtures. Stereoisomers include enantiomers and diastereomers. Stereoisomers that are not mirror images of one another are termed “diastereomers” and stereoisomers that are non-superimposable mirror images of one another are termed “enantiomers”.

As used herein, the term “tautomer” refers to a specific chemical isomerism characterized by facile interconversion of isomeric forms in equilibrium, especially by migration of a hydrogen atom. In a preferred embodiment of the present invention, a tautomer of 2-hydroxypyridine is 2-pyridone.

As used herein, the term “derivative” refers to a compound or chemical substance related structurally to another substance and theoretically derivable from it, preferably, in the context of the present invention, a compound or chemical substance that is derived from 2-HP as described herein and includes, but is not limited to, amide, ether, ester, amino, carboxyl, acetyl, and/or alcohol derivatives of the 2-HP. More preferably, a derivative of 2-HP according to the present invention may be a moiety of a ring-fused 2-pyridone molecule, most preferably a moiety of FN075. A derivative of 2-HP according to the present invention may also be any pesticide degradation product leading to the formation of 2-HP, most preferably any degradation product of chlorpyrifos, which may be specifically chlorpyrifos oxon, diethyl phosphate, 3,5,6-trichloropyridin-2-ol, diethyl-thiophosphate, phosphorothioic acid, ethanol, 3,5,6-trichloro-2-methoxy-pyridine, 2,3-dihydroxypyridine, 2,5-dihydroxpyridine, 2,5,6-trihydroxypyridine, maleamide semialdehyde, maleamic acid, pyruvic acid, aliphatic amines, carbon fragments, carboxyl, NaCl, CO₂ or NH₄CO₃. In one also most preferred embodiment, the derivative of 2-HP is a pesticide degradation product selected from the group consisting of chlorpyrifos oxon, 3,5,6-trichloropyridin-2-ol, 3,5,6-trichloro-2-methoxy-pyridine, 2,3-dihydroxypyridine, 2,5-dihydroxypyridine and 2,5,6-trihydroxypyridine. Chlorpyrifos is an organophosphate pesticide with the chemical structure or formula

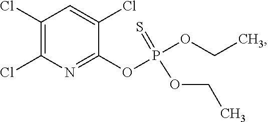 [SMILES: CCOP(=S)(OCC)Oc1nc(Cl)c(Cl)cc1Cl]

used on crops, animals, buildings and in other settings, to kill a number of pests, including insects and worms. It acts on the nervous systems of insects, by inhibiting the acetylcholinesterase enzyme. Other names for chlorpyrifos are: O,O-Diethyl-O-3,5,6-trichloropyridin-2-yl phosphorothioate, Brodan, Bolton insecticide, Chlorpyrifos-ethyl, Cobalt, Detmol UA, Dowco 179, Dursban, Empire, Eradex, Hatchet, Lorsban, Nufos, Pageant, Piridane, Scout, Stipend, Tricel or Warhawk.

The derivative of 2-hydroxypyridine can also be an archaeal cofactor (see Example 6), which is specifically a (bio)chemical compound and/or an ion, important for the catalysis of a biochemical reaction or important for metabolic reactions in a cell.

As used herein, the term “complex” refers to a molecular entity formed by loose association involving two or more component molecular entities (e.g., ionic or uncharged), or the corresponding chemical species, also meaning a chemical association of two or more species (such as ions or molecules) joined usually by weak electrostatic bonds rather than covalent bonds. It is preferred that a complex of 2-HP according to the present invention includes any complex of a moiety of a ring-fused 2-pyridone molecule, most preferably any complex of a moiety of FN075. A complex of 2-HP according to the present invention may also be preferably a complex of any pesticide degradation product leading to the formation of 2-HP, most preferably any degradation product of chlorpyrifos, which may be specifically chlorpyrifos oxon, diethyl-phosphate, 3,5,6-trichloropyridin-2-ol, diethyl-thiophosphate, phosphorothioic acid, ethanol, 3,5,6-trichloro-2-methoxy-pyridine, 2,3-dihydroxypyridine, 2,5-dihydroxpyridine, 2,5,6-trihydroxypyridine, maleamide semialdehyde, maleamic acid, pyruvic acid, aliphatic amines, carbon fragments, carboxyl, NaCl, CO₂ or NH₄CO₃. In one also most preferred embodiment, the complex of 2-HP is a complex of a pesticide degradation product selected from the group consisting of chlorpyrifos oxon, 3,5,6-trichloropyridin-2-ol, 3,5,6-trichloro-2-methoxy-pyridine, 2,3-dihydroxypyridine, 2,5-dihydroxypyridine and 2,5,6-trihydroxypyridine.

The complex of 2-hydroxypyridine can also be a complex of an archaeal cofactor, which is specifically a (bio)chemical compound, important for the catalysis of a biochemical reaction or important for metabolic reactions in a cell.

In a preferred embodiment, the inhibitor for use prevents or reduces the production of 2-hydroxypyridine.

With regard to the term “prevents” or “prevention”, it is referred to the definition given above. The term “reduces” or “reduction, as used in this embodiment and throughout the whole description means that the concentration of 2-hydroxypyridine in a sample of the subject has decreased by at least 1% 2% 3% 4% 5% 6% 7% 8% 9% 10% 11% 12% 13% 14% 15% 16% 17% 18% 19% 20% 30% 40%, 50% 60% 70% 80% , 90% , 100% , 200% , 300% , 400% , 500% , 600% , 700% , 800% , 900% , 1000%, 2000%, 3000%, 4000%, 5000%, 6000%, 7000%, 8000%, 9000%, or even 10000%, compared to the concentration of 2-hydroxypyridine in a respective sample before the inhibitor has been applied to the subject.

In a preferred embodiment according to the present invention, the inhibitor for use prevents or reduces the production of 2-hydroxypyridine by a/the methanogenic archaeon by blocking the synthesis pathway of 2-hydroxypyridine. The term “blocking” in the context of this embodiment and as used herein means the inhibition of key enzymes involved in the synthesis of 2-hydroxypyridine. In addition, the term “blocking” can also mean to reduce the amount of bacteria and/or the amount of a specific archaeon and/or the amount of more than one archaea, involved in the 2-HP synthesis by therapeutic intervention.

In an also preferred embodiment of the inhibitor for use according to the present invention, the inhibitor for use prevents or reduces the production of 2-hydroxypyridine by a methanogenic archaeon by targeting the methanogenic archaeon. The term “targeting” in the context of this embodiment and as used herein means that a given inhibitor can bind to key enzymes involved in major metabolic pathways that are important for the survival of the archaeon. This embodiment also comprises that one archaeon or more than one, for example two, three or more archaea are targeted.

In an also preferred embodiment of the inhibitor of the present invention, the inhibitor for use prevents or reduces the production of 2-hydroxypyridine by preventing the degradation of a precursor of 2-hydroxypridine, more preferably wherein the precursor of 2-hydroxypridine is derived from a pesticide, most preferably wherein the pesticide is chlorpyrifos. Such a prevention of the degradation of a precursor of 2-hydroxypyridine can for example be achieved by blocking the bacterial degradation pathway or bacterial degradation pathways of precursors of 2-hydroxypyridine, for example, by blocking the bacterial degradation pathway of such precursors as it takes place in Lactobacillus spp. Such precursors of 2-hydroxypyridine may be chlorpyrifos oxon, 3,5,6-trichloropyridin-2-ol, 3,5,6-trichloro-2-methoxy-pyridine, 2,3-dihydroxypyridine, 2,5-dihydroxypyridine or 2,5,6-trihydroxypyridine.

In a further preferred embodiment of the inhibitor for use according to the present invention, the inhibitor is selected from the group consisting of statins, preferably atorvastatin, fluvastatin, lovastatin, pitavastatin, pravastatin, rosuvastatin or simvastatin, and antibiotics, preferably rifaximin or neomycin. The term “statin” or “statins”, as used herein and in the context of the present invention, means a class of lipid-lowering medications that reduce illness and mortality in those who are at high risk of cardiovascular disease. There are various forms of statins, which include atorvastatin, fluvastatin, lovastatin, pitavastatin, pravastatin, rosuvastatin and simvastatin. The term “antibiotics”, as used herein, includes any type of antimicrobial substance active against bacteria, which includes rifaximin and neomycin. The person skilled in the art knows the respective compounds mentioned herein.

In a further preferred embodiment of the inhibitor for use according to the present invention, the 2-hydroxypyridine comprises the compound of general formula I,

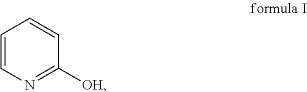

an isomer of said 2-hydroxypyridine or a mixture of isomers thereof, a derivative of said 2-hydroxypyridine or a mixture of derivatives thereof, a pharmaceutically acceptable salt of said 2-hydroxypyridine or a mixture of pharmaceutically acceptable salts thereof; or a complex of said 2-hydroxypyridine or a mixture of complexes thereof. The definitions as given above also apply for this embodiment.

It is an also preferred embodiment of the inhibitor for use according to the present invention, that the inhibitor prevents or reduces the production of 2-hydroxypyridine in the gut of said subject.

In a further preferred embodiment of the inhibitor for use according to the present invention, the inhibitor is a small molecule, preferably a small molecule having a molecular weight of less than 2000 daltons; or a biologic drug, preferably a peptide, a protein, an antibody or an antibody fragment. In a further preferred embodiment of the inhibitor for use according to the present invention, the inhibitor is a small molecule. In a further preferred embodiment of the inhibitor for use according to the present invention, the inhibitor is a small molecule having a molecular weight of less than 2000 daltons, more preferably less than 1500 daltons, even more preferably less than 1000 daltons and most preferably less than 500 daltons. In a further preferred embodiment of the inhibitor for use according to the present invention, the inhibitor is a biologic drug. In a further preferred embodiment of the inhibitor for use according to the present invention, the inhibitor is a peptide. In a further preferred embodiment of the inhibitor for use according to the present invention, the inhibitor is a protein. In a further preferred embodiment of the inhibitor for use according to the present invention, the inhibitor is an antibody. In a further preferred embodiment of the inhibitor for use according to the present invention, the inhibitor is an antibody fragment.

In an also preferred embodiment of the inhibitor for use according to the present invention, the inhibitor is for use in the treatment or prevention of a parkinsonian condition in co-treatment with other compounds, which are preferably selected from the group consisting of carbidopa-levodopa, dopamine agonists, MAO B inhibitors, catechol O-methyltransferase (COMT) inhibitors, anticholinergics, amantadine and antibiotics. As used herein, the term “carbidopa-levodopa” means the combination of Levodopa and Carbidopa, which is used in the treatment of Parkinson's disease. Levodopa is in a class of medications called central nervous system agents. It works by being converted to dopamine in the brain. Carbidopa is in a class of medications called decarboxylase inhibitors. It works by preventing levodopa from being broken down before it reaches the brain. As used herein, the term “dopamine agonist(s)” means a molecule or molecules that exert(s) their antiparkinsonian effects by acting directly on dopamine receptors and mimicking the endogenous neurotransmitter. Dopamine agonists include ergoline (derivatives of an alkaloid called ergot) and non-ergoline agonists. Ergoline agonists such as bromocriptine, cabergoline, and pergolide are first-generation agents, whereas non-ergoline derivatives such as pramipexole, ropinirole, rotigotine, and apomorphine are second-generation medications. As used herein, the term “MAO B inhibitors” means monoamine oxidase-B inhibitors and is a class of medications that are used to treat the symptoms of Parkinson's disease. MAO B inhibitors include Rasagiline, Selegiline and Safinamide. As used herein, the term “catechol O-methyltransferase (COMT) inhibitors” means a class of medications that are used along with carbidopa-levodopa therapy in the treatment of Parkinson's disease. This class of medication includes Entacapone and Tasmar. As used herein, the term “anticholinergics” means a class of medications that is used to treat the symptoms of Parkinson's disease, particularly the tremor. This class of medication includes benzotropine mesylate and trihexyphenidyl. As used herein, the term “amantadine” means a glutamate antagonist drug that is prescribed to treat Parkinson's disease. The preferred antibiotic for this embodiment includes rifaximin or neomycin.

In a further preferred embodiment of the inhibitor for use according to the present invention, the subject is administered a therapeutically effective amount of the inhibitor, preferably at least 0.1 mg. In a further preferred embodiment of the inhibitor for use according to the present invention, the subject is administered a therapeutically effective amount of the inhibitor of at least 0.2 mg. In a further preferred embodiment of the inhibitor for use according to the present invention, the subject is administered a therapeutically effective amount of the inhibitor of at least 0.3 mg. In a further preferred embodiment of the inhibitor for use according to the present invention, the subject is administered a therapeutically effective amount of the inhibitor of at least 0.4 mg. In a further preferred embodiment of the inhibitor for use according to the present invention, the subject is administered a therapeutically effective amount of the inhibitor of at least 0.5 mg. In a further preferred embodiment of the inhibitor for use according to the present invention, the subject is administered a therapeutically effective amount of the inhibitor of at least 0.6 mg. In a further preferred embodiment of the inhibitor for use according to the present invention, the subject is administered a therapeutically effective amount of the inhibitor of at least 0.7 mg. In a further preferred embodiment of the inhibitor for use according to the present invention, the subject is administered a therapeutically effective amount of the inhibitor of at least 0.8 mg. In a further preferred embodiment of the inhibitor for use according to the present invention, the subject is administered a therapeutically effective amount of the inhibitor of at least 0.9 mg. In a further preferred embodiment of the inhibitor for use according to the present invention, the subject is administered a therapeutically effective amount of the inhibitor of at least 1 mg. In a further preferred embodiment of the inhibitor for use according to the present invention, the subject is administered a therapeutically effective amount of the inhibitor of at least 2 mg. In a further preferred embodiment of the inhibitor for use according to the present invention, the subject is administered a therapeutically effective amount of the inhibitor of at least 3 mg. In a further preferred embodiment of the inhibitor for use according to the present invention, the subject is administered a therapeutically effective amount of the inhibitor of at least 5 mg. In a further preferred embodiment of the inhibitor for use according to the present invention, the subject is administered a therapeutically effective amount of the inhibitor of at least 10 mg.

In an also preferred embodiment of the inhibitor for use according to the present invention, the subject is administered a dosage of the inhibitor in the range of 0.1 to 10 mg per day. In an also preferred embodiment of the inhibitor for use according to the present invention, the subject is administered a dosage of the inhibitor in the range of 0.2 to 10 mg per day. In an also preferred embodiment of the inhibitor for use according to the present invention, the subject is administered a dosage of the inhibitor in the range of 0.2 to 7 mg per day. In an also preferred embodiment of the inhibitor for use according to the present invention, the subject is administered a dosage of the inhibitor in the range of 0.5 to 5 mg per day. In an also preferred embodiment of the inhibitor for use according to the present invention, the subject is administered a dosage of the inhibitor in the range of 1 to 5 mg per day. In an also preferred embodiment of the inhibitor for use according to the present invention, the subject is administered a dosage of the inhibitor in the range of 2 to 5 mg per day.

In a further preferred embodiment of the inhibitor for use according to the present invention, the parkinsonian condition is selected from the group consisting of Parkinson's disease, Parkinsonism, Rapid Eye Movement Sleep Behavior Disorder and Parkinson-plus syndrome. The definitions as given above also apply for this embodiment.

The present invention also relates to a kit comprising the inhibitor for use according to the present invention.

In a preferred embodiment of the kit according to the present invention, the kit further comprises a pharmaceutically acceptable carrier and/or an antiparkinsonian agent, preferably said antiparkinsonian agent is one or more of the following: L-DOPA, deprenyl, apomorphine, an anticholinergic agent, further preferably said anticholinergic agent is selected from the group consisting of benzhexol and orphenadrine. As used herein, the term “L-DOPA” means Levodopa as described above. As used herein, the term “deprenyl” means Selegiline, specific monoamine oxidase subtype B inhibitor. As used herein, the term “apomorphine” means a dopamine agonist as described above. As used herein, the term “anticholinergic agent” means a class of medications that are used to treat the symptoms of Parkinson's disease, particularly the tremor, as described above, including benzotropine mesylate and trihexyphenidyl. As used herein, the term “benzhexol” means an antiparkinsonian agent of the antimuscarinic class. As used herein, the term “orphenadrine” means an anticholinergic drug of the ethanolamine antihistamine class.

Further, the present invention also relates to a pharmaceutical composition comprising the inhibitor for use according to the present invention.

The present invention also relates to a method of screening for an inhibitor for use according to the present invention and as described elsewhere herein, wherein the method comprises (i) contacting a compound of interest with an iron-containing hydrogenase of a methanogenic archaeon, (ii) measuring inhibitor function of the compound of interest for the iron-containing hydrogenase of the methanogenic archaeon, wherein an increase of inhibition of said iron-containing hydrogenase compared to the iron-containing hydrogenase before contacting of step (i) indicates that the compound of interest serves as an inhibitor for the iron-containing hydrogenase, or wherein a decrease or maintenance of inhibition of said iron-containing hydrogenase compared to the iron-containing hydrogenase before contacting of step (i) indicates that the compound of interest does not serve as an inhibitor for the iron-containing hydrogenase.

The term “measuring inhibitor function” may include the following means, known to the person skilled in the art, which are specifically in vitro determination of the half maximum inhibitory concentration (IC50), growth inhibition power (GI50), apoptosis induction, neurotoxicity and enzymatic binding assays.

The term “increase of inhibition” means that the enzymatic reaction, which is catalyzed by the iron-containing hydrogenase is inhibited or cannot take place in that amount any more, compared to the enzymatic reaction before applying the compound of interest.

The term “decrease of inhibition” means that the enzymatic reaction, which is catalyzed by the iron-containing hydrogenase is not inhibited or not that much inhibited or cannot take place in in the same amount, compared to the enzymatic reaction before applying the compound of interest.

This method of screening may further, additionally or alternatively comprise or include that the target may be a bacterial degradation pathway. For example, chlorpyrifos is the main source of the metabolite 3,5,6-trichloro-2-pyridinol, which in turn might be transformed into 2-HP, probably via dechlorination by bacteria such as Lactobacillus spp. This is a pathway which can be directed according to the present invention to screen for an inhibitor for use according to present invention and for determining a compound of interest that may serve as an inhibitor for the iron-containing hydrogenase.

The present invention also relates to a method for determining whether or not a subject is susceptible to a treatment with an inhibitor for use according to the present invention, (i) comprising determining the concentration of 2-hydroxypyridine in a sample obtained from the gut of said subject, compared to a control sample of the same subject.

The control sample of the same subject may be obtained from the subject a certain time before step (i), more preferably wherein certain time means at least 1, 2, 3, 4, 5, 6, 7, 8, 9, 10 or 11 months, even more preferably at least 1 year, 2 years, 3 years, 4 years, 5 years, 6 years, 7 years, 8 years, 9 years or even 10 years, before step (i).

In a preferred embodiment of said method for determining whether or not a subject is susceptible to a treatment with an inhibitor for use according to the present invention, the susceptibility is positive (is present) or is assessed as being positive, if the concentration of 2-hydroxypyridine has decreased compared to the control sample of the same subject or has maintained the same compared to the control sample of the same subject. The term “susceptibility is positive” means in the context of the present invention that an improvement of the disease status of a/the subject or patient has occurred, e.g. the parkinsonian condition is lower or not that much present, distinct or strong anymore.

The concentration of 2-hydroxypyridine has decreased compared to the control sample of the same subject means, in the context of said method, that the concentration of 2-hydroxypyridine in the sample of the subject has decreased by at least 11%, 12%, 13%, 14% 15% 16% 17% 18% 19% 20% 30% 40%, 50% 60% 70% 80% 90% 100%, 200%, 300%, 400%, 500%, 600%, 700%, 800%, 900%, 1000%, 2000%, 3000%, 4000%, 5000%, 6000%, 7000%, 8000%, 9000%, or even 10000%, compared to the concentration of 2-hydroxypyridine in the control sample.

The concentration of 2-hydroxypyridine has maintained the same compared to the control sample of the same subject means that the concentrations only deviate by +10% or −10%.

It is also preferred for said method for determining whether or not a subject is susceptible to a treatment with an inhibitor for use according to the present invention, that the susceptibility is negative (is not present) or is assessed as being negative, if the concentration of 2-hydroxypyridine has increased compared to the control sample of the same subject, more preferably wherein the concentration of 2-hydroxypyridine has significantly increased compared to the control sample of the same subject. The term “susceptibility is negative” means in the context of the present invention that a worsening of the disease status of a/the subject or patient has occurred, e.g. the parkinsonian condition is much more present, distinct, strong or evident.

The concentration of 2-hydroxypyridine has increased compared to the control sample of the same subject means, in the context of the present invention, that the concentration of 2-hydroxypyridine in the sample of the subject has increased by at least 1%, 2% 3% 4% 5% 6% 7% 8% 9% 10% 11%, 12% 13% 14% 15% 16% 17% , 18% , 19% , 20% , 30% , 40% , 50% , 60% , 70% , 80% , 90% , 100% , 200% , 300% 400% , 500% , 600% , 700% , 800% , 900% , 1000% , 2000% , 3000% , 4000% , 5000 6000%, 7000%, 8000%, 9000%, or even 10000%, compared to the concentration of 2-hydroxypyridine in the control sample.

A significant increase in this embodiment is given, when the concentration of 2-hydroxypyridine in the sample of the subject has increased by at least 30%, 40%, 50%, 60%, 70%, 80%, 90%, 100%, 200%, 300%, 400%, 500%, 600%, 700%, 800%, 900%, 1000%, 2000%, 3000%, 4000%, 5000%, 6000%, 7000%, 8000%, 9000%, or even 10000%, compared to the concentration of 2-hydroxypyridine in the control sample. In a more preferred embodiment, a significant increase is given, when the concentration of 2-hydroxypyridine in the sample of the subject has increased by at least 30%, compared to the concentration of 2-hydroxypyridine in the control sample.

It is preferred for the method whether or not a subject is susceptible to a treatment with an inhibitor for use according to the present invention, that the concentration of 2-hydroxypyridine is determined by mass spectrometry or NMR.

The term “mass spectrometry”, as used in the context of the present invention, includes any method known to the person skilled in the art, wherein the mass as a value can be determined. This may be, for example, a device including an ion source for generating mainly molecular or pseudo-molecular ions. The ion source may be an atmospheric pressure ionization source, e.g. an Electrospray Ionisation (“ESI”)-ion source, a chemical-atmospheric pressure ionization (“APCI”)-ion source, an Atmospheric Pressure Photoionization (“APPI”)-ion source or an atmospheric pressure ion source. Alternatively, the ion source may comprise a non-atmospheric pressure ionization source, for example, a Fast Atom Bombardment (“FAB”)-ion source, a Liquid Secondary Ions Mass Spectrometry (“LSIMS”)-ion source, a matrix-assisted-laser desorption ionization (“MALDI”)-ion source, a matrix assisted Laser Desorption Ionisation-(“MALDI”)-ion source in combination with a collision cell for collision cooling ions or a Laser Desorption Ionisation (“LDI”)-ion source.

The term “NMR”, as used in the context of the present invention, includes any method known to the person skilled in the art using an NMR apparatus. Such may contain in a statoric frame magnetic means, radio frequency (RF) means comprising excitation circuits, an emitting coil and RF receiving means. Located in a central hole of this statoric frame, this apparatus contains positioning means for holding the sample or the object to analyze. In some cases, especially in solid state NMR, the means for holding the sample have means for spinning it, and sometimes extra means for tilting the spinning axis. The spinning part is called the rotor. It has a cylindrical shape and wears at one end tiny turbo blades driven by air jets. The rotor is made of a material transparent for magnetic fields, generally made of ceramics. It may be filled with the sample to analyze, but when this sample is small, it wears an internal sample container, mechanically centered and whose axis is parallel to the mechanical axis of the rotor. It is necessary to underline that frequently, in NMR literature, the sample container is abusively called in short “sample” instead of “sample container”. As known for many years, in NMR spectroscopy and/or imaging, the sample—be it an object or a subject—is placed inside a strong static and very homogeneous magnetic field B₀. In a quantum description, nuclear spins (assuming that they have a quantum number I=½) can be parallel or anti-parallel with respect to the static magnetic field B₀. Each of these two states has a different energy in the presence of B₀. These energy levels are named Zeeman energy levels, and the spins can absorb energy in the radio-frequency range, to undergo transitions between their two states. In a classical description, the magnetic moments of the nuclear spins process around the static magnetic field. The frequency ω_{L} of the precession (called “Larmor precession”) is roughly proportional to the static magnetic field, also depending on the local chemical environment and can be used to probe molecular structure and dynamics. In order to absorb energy and induce transitions, an oscillating magnetic field B₁ needs to be applied. This field is produced by antennas (i.e. coils) surrounding the object or subject. This field is oscillating at the Larmor frequency (resonance condition) and can be applied for time delays long enough to perturb the magnetization and rotate it at various angles. The NMR method used for determining the specific concentration of 2-hydroxypyridine may include conducting an ¹H-NMR, HSQCs, TOCSY, COSY, high-pressure-NMR-spectrum or any further NMR-experiment, known to a person skilled in the art for determining concentrations.

In a more preferred embodiment, untargeted Gas Chromatography-Mass Spectrometry (GC-MS) with Electron Impact ionization (EI) source could be used for determining the concentration of 2-HP. The result thereof is to identify, validate and quantify 2-HP. The analysis by GC-MS produces a mass intensity spectrum, wherein the peaks of it represent various components of said sample, each component having a characteristic mass-to-charge ratio (m/z) and retention time (RT). The peak representing 2-HP may be compared to a corresponding peak from another spectrum, e.g., from the control sample as defined herein, to obtain a relative measurement. A normalization technique (analytical standard) is used when a quantitative measurement is desired. Such, may for example be carried out like described in the following: Normalizing is achieved by an internal standard, i.e. a ¹³C-labeled compound that differs from endogenous compounds to normalize the data and to evaluate the GC-MS run performance. To enable a quantitative measurement, one has to perform a calibration curve, i.e. additional samples in the GC-MS data acquisition run with increasing concentrations of 2-HP. The concentration of 2-HP is thereby calculated upon linear regression based on (1) the linear function of the calibration curve and (2) the measured value of 2-HP in the samples of interest.

Unless otherwise specified, the terms used herein have their common general meaning as known in the art.

It is noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

The term “and/or”, wherever used herein, includes the meaning of “and”, “or” and “all or any other combination of the elements connected by said term”.

The term “less than” or in turn “more than” does not include the concrete number.

For example, “less than 20” means less than the number indicated. Similarly, “more than” or “greater than” means more than or greater than the indicated number, e.g. “more than 80%” means more than or greater than the indicated number of 80%.

Throughout this specification and the claims which follow, unless the context requires otherwise, the word “comprise”, and variations such as “comprises” and “comprising”, will be understood to imply the inclusion of a stated integer or step or group of integers or steps, but not the exclusion of any other integer or step or group of integer or step. When used herein the term “comprising” can be substituted with the term “containing” or “including” or sometimes, when used herein, with the term “having”. When used herein, “consisting of” excludes any element, step, or ingredient not specified.

The term “including” means “including but not limited to”. “Including” and “including but not limited to” are used interchangeably.

It should be understood that this invention is not limited to the particular methodology, protocols, material, reagents, and substances, etc., described herein and as such can vary. The terminology used herein is for the purpose of describing particular embodiments only, and is not intended to limit the scope of the present invention, which is defined solely by the claims.

All publications cited throughout the text of this specification (including all patents, patent application, scientific publications, instructions, etc.), whether supra or infra, are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

The content of all documents and patent documents cited herein is incorporated by reference in their entirety.

A better understanding of the present invention and of its advantages will be had from the following examples, offered for illustrative purposes only. The examples are not intended to limit the scope of the present invention in any way.

### EXAMPLES OF THE INVENTION

In order that the invention may be readily understood and put into practical effect, some aspects of the invention are described by way of the following non-limiting examples.

Supplementary Methods:

Stool Extraction and GC-MS Measurement

Stool samples were collected and snap-frozen in liquid nitrogen without any additives or stabilizers. Storage was performed within 1 h post-collection in −80° C. and shipment was on dry ice. Stool samples were homogenized using a cryogenic grinder (6875D Freezer/Mill®-SPEX). To extract polar metabolites, 500 μL of MilliQ water was added to 50 mg fecal matter. Then, samples were homogenized using Precellys24 homogenizer (Bertin Technologies): 6000 rpm, 1×30 sec at 0 to 5° C. Then, untargeted GC-MS analysis (Quadrupole, EI Source) was performed for the identification, validation and quantification by external calibration measurements with analytical standards (Sigma Aldrich, 41152). Further sample preparation, measurement parameters and data analysis were described in Glaab et al. (2019).

### Example 1: Alpha-synuclein-yeast Experiment

Yeast Strains, which have been Used, are Shown in Table 1 Below.

TABLE 1

| | | |
|---|---|---|
| W303 | MATa ura3-52 trp1Δ2 leu2-3_112 his3-11 | Parental strain |
| | ade2-1 can 1-100 | |
| HiTox | W303 pdr5Δ::KanMX4, pAG306GAL-SNCA- | Derived strain |
| | EGFP, pAG304GAL-SNCA-EGFP | |
| Control | W303 pdr5Δ::KanMX4, pAG306GAL-ccdB- | Derived strain |
| | EGFP, pAG304GAL-ccdB-EGFP | |

Yeast Cultivation Media

Yeast cells were grown in synthetic complement (SC) media containing 6.7 g/L yeast nitrogen base without amino acids, 5 g/L ammonium sulfate supplemented with 2 g/L SC. Media was autoclaved and the carbon source (8% raffinose or galactose) was added to a final concentration of 2%.

Yeast Cultivation and Phenotyping

Four fresh single colonies of the Hitox strain and its respective control strain were inoculated from SC-2% glucose plates into 5 mL SC-2% raffinose and incubated overnight with shaking (200 rpm) at 30° C. After ≈20 h, overnight cultures were diluted to OD 0.5 and 2 μL of the yeast cultivation was added to 78 μL drug containing media to a final optical density of 0.0125 in a 384-well microplate. 2-HP was diluted in SC-2% raffinose/galactose and tested at different concentrations (1-100 mM). Finally, the plates are measured in a microplate reader (TECAN™ Infinite M200Pro), at an interval of 10 minutes during 72 h at 30° C. Yeast growth phenotyping was performed as previous described (Jung et al. 2015). Final biomass was corrected using the GATHODE software (Jung et al., 2015). The OD₆₀₀ at 48 h was taken for the quantification.

### Example 2: Dopaminergic Neuron Experiment

Enteric Neurons Cultivation and Phenotyping

Enteric neurons were cultured in differentiation medium, consisting on Neurobasal (Invitrogen, 21103049) supplemented with 1:200 N2 (Invitrogen, 17502048), 1:100 L-Glutamine (Invitrogen, 25030-024), 1:100 B-27 without Vitamin A (Life Technologies, 12587-010), 1:100 Penicillin/Streptomycin (Invitrogen, 15140122), 25 ng/mL GDNF (Peprotech, 450-10) and 100 μM Ascorbic Acid (Sigma, A5960). Cells were cultured for 21 days in a 6-well plate, then detached with Accutase (Sigma, A6964), and replated into a 96-well plate. Cells were maintained under differentiating conditions until day 31, when they were treated with increasing concentrations of 2-hydroxypyridine (Sigma, H56800). Tested concentrations were as follows: 1 μM, 3 μM, 6 μM, 10 μM, 30 μM, 60 μM, 100 μM, 300 μM, 600 μM, 1 mM and 3 mM.

2-Hydroxypyridine was reconstituted to 10 mM in the differentiation medium described above.

Cells were treated for 24 h, then cell viability was assessed performing a Tetrazolium (MTT) assay. Thiazolyl Blue tetrazolium Bromide (Sigma, M5655) was reconstituted to a 5 mg/mL solution in differentiation media and filtered. 10 μL of the solution were then added to every well containing 100 μL of media. Plate was incubated for 2 h in a normal incubator (37° C., 5% CO₂). Media containing MTT solution was removed and 100 μL of DMSO (Sigma, D8418) were added to lyse the cells. Cells were disaggregated by pipetting vigorously and absorbance was read at 570 nm, using the microplate Cytation 5M reader (Biotek).

Alpha-Synuclein Aggregation Staining in Enteric Neurons

Cells fixed in 4% paraformaldehyde (Merck Millipore, 1004965000) for 15 min at RT and then washed 3× for 5 min with PBS at RT. Prior to immunostaining, a permeabilization step was performed using 0.05% Triton-X100 solution in PBS for 10 min at 4° C. Cells were then blocked for 1 h at RT with 10% FBS in PBS. Incubation with α-synuclein antibody (NOVUS biologicals, NBP1-05194, 1:1000), α-synuclein filament antibody (Abcam, ab20953, 1:5000) and TUJ1 (Millipore, AB9354, 1:600) was done overnight at 4° C. in blocking buffer. The following day, cells were washed with PBS 3× for 5 min at RT. Then, incubation with the corresponding secondary antibodies, Alexa Fluor anti-chicken 488 (Invitrogen, A-11039, 1:1000), Alexa Fluor anti-mouse IgG1 647 (Invitrogen, A-21240, 1:1000) and Alexa Fluor anti-rabbit 568 (Invitrogen, A11036, 1:1000) was performed for 2 h at RT in blocking buffer. Hoechst 33342 solution (Invitrogen, 62249, 1:1000) was added during this step to stain the nuclei. After incubation, cells were washed 3× with PBS and imaged directly afterwards.

### Example 3: In Silico Target Prediction of 2-HP

To identify putative protein targets of 2-HP, an inverse in silico screening was performed. This analysis is based on in silico docking and binding affinity prediction with proteins. In general, these methods have a high sensitivity (recall around 80%) but a lower specificity (precision between 20 and 40%) and require further filtering and in vitro validation to remove false positives.

Multiple putative targets in relation with Parkinson's disease have been identified such as dopamine beta-hydroxylase (DBH) and cathechol o-methyltransferase (COMT) both involved in the dopamine metabolism. For DBH, a crystal structure was available which enabled a stronger docking and binding affinity prediction analysis, hence confirming that 2-HP has at least a weak affinity for this target. In addition, DBH polymorphisms have already been shown in the literature to be tightly linked to PD pathology.

### Example 4: PD Mouse Model

By chronically (ad libitum) exposing the animals to 2-HP via the drinking water, an increased accumulation up to aggregation of alpha-synuclein in ENS of transgenic mice (Thy1-Syn14 males) is seen. Newly formed oligomeric forms are then propagated to the CNS via the vagus nerve. Subsequently, an exacerbation of motor symptoms and triggering of PD-like pathology is observed.

Chemically, 2-HP is determined to be acute toxic and irritant. Its LD₅₀ (oral administration) in rats is at 124 mg/kg (Section 11, FDS by Sigma-Aldrich). To control toxicity and set an upper threshold level for the chronic exposure study, the ideal concentration in the animals' drinking water (ad libitum) is determined by testing three different concentrations. As reference, the LD₅₀ value determined in rats is used as a threshold for an entire day. The animals, of an average weight of 28 g, take in 3.472 g of 2-HP per day. The second factor to account for is the volume of water a C57BI6/j mouse drinks per day, which varies between 5 mL and 8 mL. With an average of 6.5 mL water intake per day, the maximum concentration the inventors use is 695 μg/mL of dissolved 2-HP. However, lower doses are aimed for:

TABLE 2

| Dose 1 (⅓) | Dose 2 ( 1/7) | Dose 3 ( 1/10) |
|---|---|---|
| 190 μg/mL | 80 μg/mL | 55 μg/mL |

These 3 doses are tested on a total of 48 animals. 24 wild-type and 24 transgenic animals are distributed amongst 12 groups. It is essential to have both genotypes in the study, since it cannot be foreseen how human wild-type alpha-synuclein and 2-HP interact in an in vivo model. Further, short-term and long-term effects are analysed. Additionally, this allows to proceed with the post-mortem analysis of the 14 days cohort, allowing to gather information of the early effects of 2-HP on an in vivo system. The following design is used:

TABLE 3

| | | | Dose 1 | Dose 2 | Dose 3 | |
|---|---|---|---|---|---|---|
| Genotype | Time points | | (⅓) | ( 1/7) | ( 1/10) | Number |
| WT | 14 | days | 4 | 4 | 4 | 12 |
| TG | 14 | days | 4 | 4 | 4 | 12 |
| WT | 8 | weeks | 4 | 4 | 4 | 12 |
| TG | 8 | weeks | 4 | 4 | 4 | 12 |

| | |
|---|---|
| TOTAL | 48 |

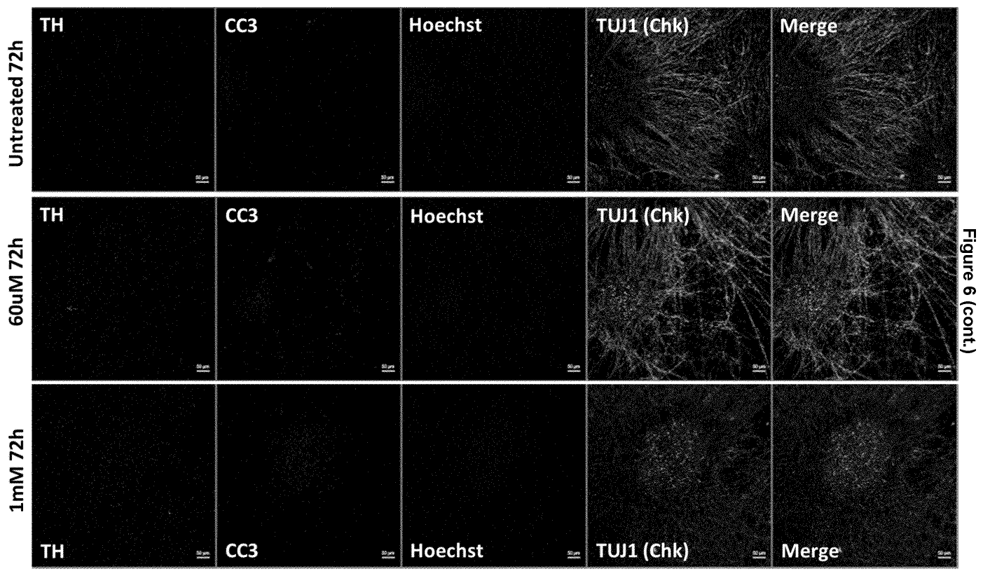

The procedure used is further given in detail in FIG. 7 shown below.

In the in-life phase, all animals are checked regularly. Overall behavior in the cage is checked daily. Weight is checked twice a week. Blood (tail vein) and stool samples are taken once a week to check 2-HP levels.

The first cohort is euthanized by anesthesia (medetomidine (1 mg/kg) & ketamine (150 mg/kg), 100 μL/10 g mouse weight) and transcardial perfusion after 14 days. Blood is harvested using the cardiac puncture procedure (0.8-1 mL) in parallel with the transcardial perfusion. All other organs and tissues (see figure above) will be collected post-mortem and subsequently prepared for histological analysis by a veterinary pathologist.

### Example 5: 2-HP Upregulates Proinflammatory Cytokine IL-1b in a Murine Macrophage Model

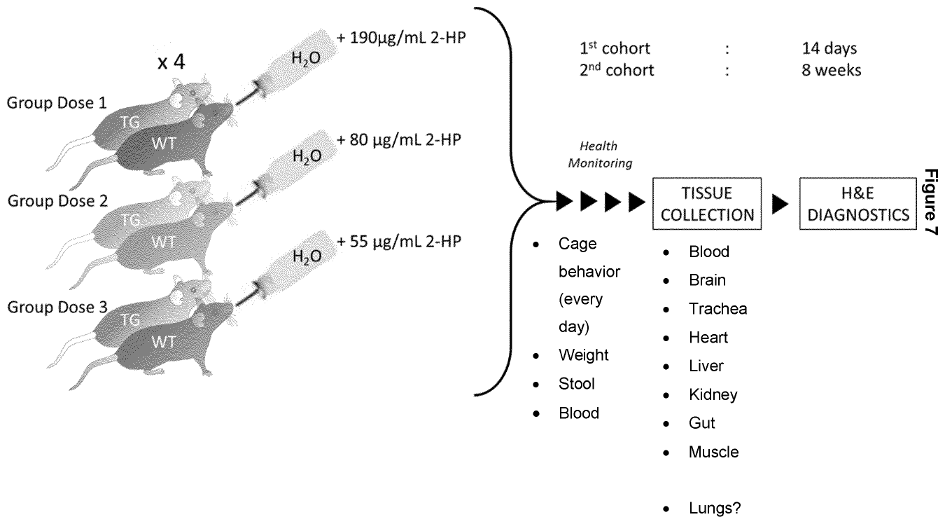

The inventors have tested whether 2-HP can act as signal 1 in the activation of the NLRP3 inflammasome in murine bone-barrow derived macrophages (BMDMs). NLRP3 inflammasome activation is thought to play a role in the pathogenesis of neurodegenerative disorders, and increased levels of this inflammasome and of the related cytokine IL-1b have been detected in Parkinson's patients (Guan & Han, 2020). In these experiments, the cells were grown in BMDM growth media and treated with 2-hydroxypyridine (2.5 and 10 mM) for up to 72 hours. Inflammasome activation was evaluated with qPCR for IL-1b expression (normalized to β-actin), considering the fold change relative to 0 mM 2-HP of each time point. There was a 3.2-fold increase in IL-1b mRNA after 3 hours of 10 mM 2-HP treatment, and 1.5-fold increase after 24 hours (FIG. 8).

### Example 6: Archaeal Enzyme Linked to 2-HP is Present in Sequence Data From Stool Samples

To verify that 5,10-methenyltetrahydromethanopterin hydrogenase, the archaeal enzyme that has a 2-HP derivative cofactor, is present in the MiBiPa stool samples, the inventors have mined metagenomic and metatranscriptomic sequence reads to look for this enzyme. The inventors were able to detect it in the samples, and it was present in a higher proportion of samples from PD and RBD patients than from control subjects (see Table 4).

TABLE 4
Percent (%) of stool samples that contain the archaeal enzyme
5,10-methenyltetrahydromethanopterin hydrogenase.

| Type of data | Ctrl | PD | RBD |
|---|---|---|---|
| Metagenomic | 36.73 | 48.94 | 40.74 |
| Metatranscriptomic | 40.82 | 51.06 | 48.15 |

### Example 7: Genus Methanobrevibacter is More Abundant in PD Compared to Controls in a Large Cohort

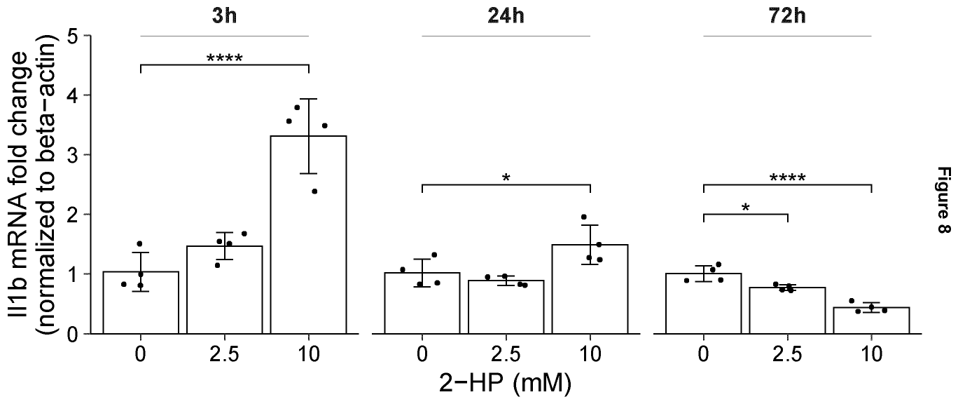

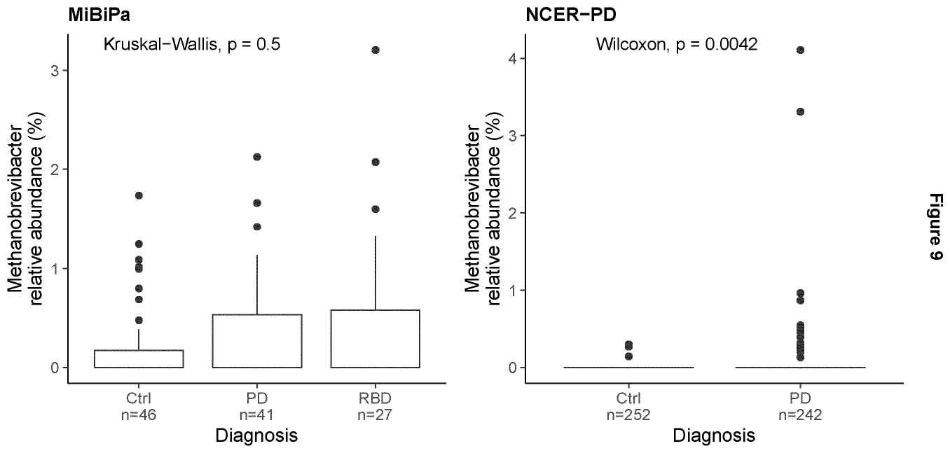

As 2-HP levels are higher in PD and RBD patients than in controls, and correlated with Methanobrevibacter abundance, the inventors tested whether Methanobrevibacter is more abundant in PD and RBD patients than in controls. Comparing 16S rRNA gene amplicon data from MiBiPa subjects, there was a numerical difference between groups. Considering similar data from a larger cohort, NCER-PD (Baldini et al. 2020), there was a statistically significant difference between the PD and control groups for this genus (FIG. 9). The difference was also significant if the two cohorts were combined and tested together (p=0.003; fdr=0.015).

### Example 8: 2-HP is Detectable in Archaeal Cell Cultures

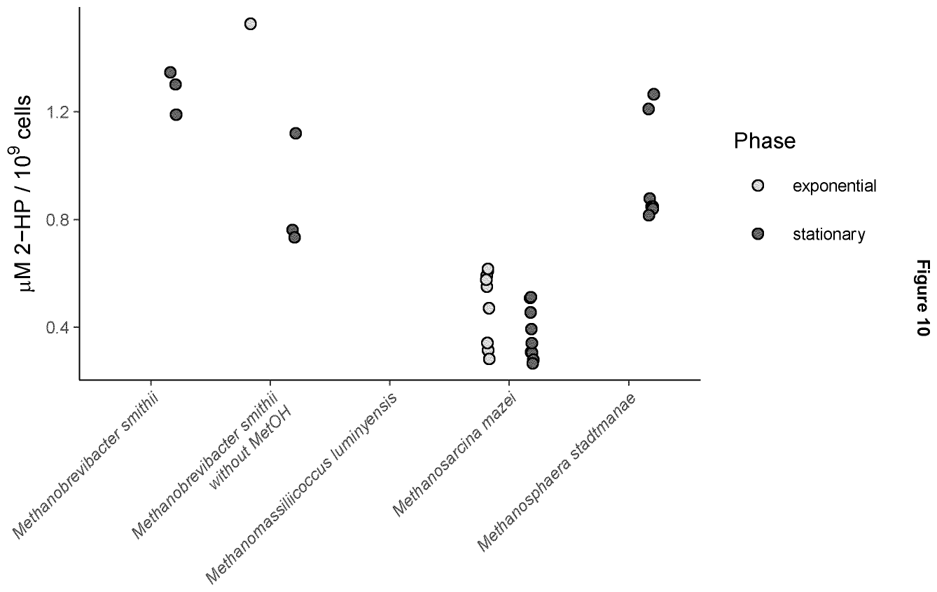

To validate that 2-HP is linked to archaeal metabolism and for showing correlations between 2-HP concentration and Methanobrevibacter abundance in stool samples, the inventors cultured several archaeal species and measured the concentrations of 2-HP in the cultures with targeted GC/MS. 2-HP was detectable in these samples, with the highest cell count normalized concentrations in cell pellets of Methanobrevibacter smithii and Methanosphaera stadtmanae, two archaeal species present in the human gut (FIG. 10).

### Example 9: In Vivo Results Show Sensorimotor Deficits Due to 2-HP Treatment

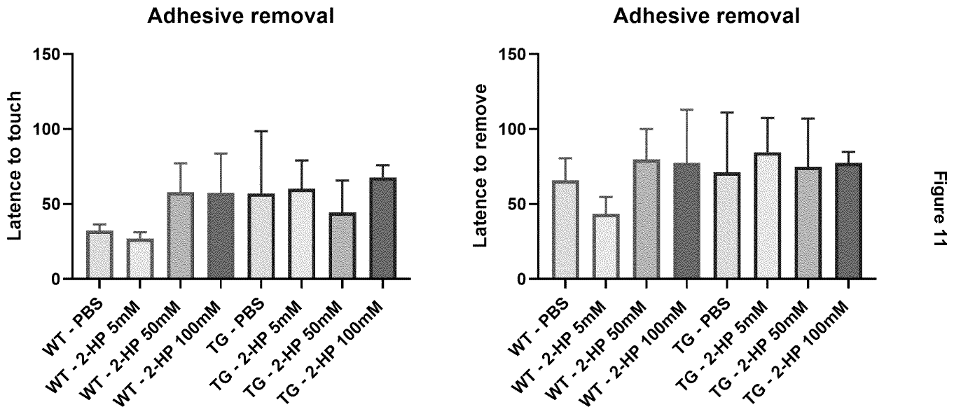

The in vivo work testing for effects of 2-HP in a mouse model lead to results from a small-scale 15-day experiment. In this experiment, 2-HP was injected at several concentrations (5, 50 and 100 mM) into the brain of transgenic mice overexpressing human alpha-synuclein, and of wildtype control mice. Behavioral assays at 15 days post-injection showed that wildtype mice treated with higher 2-HP concentrations perform worse in the adhesive removal test (a measure of sensorimotor function; Fleming et al. 2013) than those treated with control solution (PBS) (FIG. 11).

The present invention further relates to the following items:
1. An inhibitor of an iron-containing hydrogenase of a methanogenic archaeon for use in the treatment or prevention of a parkinsonian condition,
wherein said archaeon is in the gut of a subject and is characterized by the production of 2-hydroxypyridine.
2. Inhibitor for use according to item 1, wherein the inhibitor prevents or reduces the production of 2-hydroxypyridine.
3. Inhibitor for use according to item 2, wherein the inhibitor prevents or reduces the production of 2-hydroxypyridine by the methanogenic archaeon by blocking the synthesis pathway of 2-hydroxypyridine.
4. Inhibitor for use according to item 2 or item 3, wherein the inhibitor prevents or reduces the production of 2-hydroxypyridine by the methanogenic archaeon by targeting the methanogenic archaeon.
5. Inhibitor for use according to any one of the preceding items, wherein the inhibitor prevents or reduces the production of 2-hydroxypyridine by preventing the degradation of a precursor of 2-hydroxypridine, preferably wherein the precursor of 2-hydroxypridine is derived from a pesticide, more preferably wherein the pesticide is chlorpyrifos.
6. Inhibitor for use according to any one of the preceding items, wherein the inhibitor is selected from the group consisting of statins, preferably atorvastatin, fluvastatin, lovastatin, pitavastatin, pravastatin, rosuvastatin or simvastatin, and antibiotics, preferably rifaximin and neomycin.
7. Inhibitor for use according to any one of the preceding items, wherein 2-hydroxypyridine comprises the compound of general formula I,

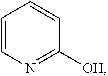

an isomer of said 2-hydroxypyridine or a mixture of isomers thereof;
a derivative of said 2-hydroxypyridine or a mixture of derivatives thereof;
a pharmaceutically acceptable salt of said 2-hydroxypyridine or a mixture of pharmaceutically acceptable salts thereof; or
a complex of said 2-hydroxypyridine or a mixture of complexes thereof.
8. Inhibitor for use according to any one of the preceding items, wherein the inhibitor prevents or reduces the production of 2-hydroxypyridine in the gut of said subject.
9. Inhibitor for use according to any one of the preceding items, wherein the inhibitor is a small molecule, preferably a small molecule having a molecular weight of less than 2000 daltons; or a biologic drug, preferably a peptide, a protein, an antibody or an antibody fragment.
10. Inhibitor for use according to any one of the preceding items, wherein the inhibitor is for use in the treatment or prevention of a parkinsonian condition in co-treatment with other compounds, which are preferably selected from the group consisting of carbidopa-levodopa, dopamine agonists, MAO B inhibitors, catechol O-methyltransferase (COMT) inhibitors, anticholinergics, amantadine and antibiotics.
11. Inhibitor for use according to any one of the preceding items, wherein the subject is administered a therapeutically effective amount of the inhibitor, preferably at least 0.1 mg.
12. Inhibitor for use according to any one of the preceding items, wherein the subject is administered a dosage of the inhibitor in the range of 0.1 to 1.0 mg per day.
13. Inhibitor for use according to any one of the preceding items, wherein the parkinsonian condition is selected from the group consisting of Parkinson's disease, Parkinsonism, Rapid Eye Movement Sleep Behavior Disorder and Parkinson-plus syndrome.
14. Kit comprising the inhibitor for use according to any one of the preceding items.
15. The kit according to item 14, further comprising a pharmaceutically acceptable carrier and/or an antiparkinsonian agent,
preferably said antiparkinsonian agent is one or more of the following: L-DOPA, deprenyl, apomorphine, an anticholinergic agent, further preferably said anticholinergic agent is selected from the group consisting of benzhexol and orphenadrine.
16. Pharmaceutical composition comprising the inhibitor for use according to any one of items 1 to 13.
17. Method of screening for an inhibitor for use according to any one of items 1 to 13, wherein the method comprises
(i) contacting a compound of interest with an iron-containing hydrogenase of a methanogenic archaeon,
(ii) measuring inhibitor function of the compound of interest for the iron-containing hydrogenase of the methanogenic archaeon,
wherein an increase of inhibition of said iron-containing hydrogenase compared to the iron-containing hydrogenase before contacting of step (i) indicates that the compound of interest serves as an inhibitor for the iron-containing hydrogenase, or
wherein a decrease or maintenance of inhibition of said iron-containing hydrogenase compared to the iron-containing hydrogenase before contacting of step (i) indicates that the compound of interest does not serve as an inhibitor for the iron-containing hydrogenase.
18. Method for determining whether or not a subject is susceptible to a treatment with an inhibitor for use according to any one of the items 1 to 13, (i) comprising determining the concentration of 2-hydroxypyridine in a sample obtained from the gut of said subject, compared to a control sample of the same subject.

### REFERENCES

Baldini F, Hertel J, Sandt E, et al. Parkinson's disease-associated alterations of the gut microbiome predict disease-relevant changes in metabolic functions. BMC Biol. 2020;18(1):62. Published 2020 Jun. 9. doi:10.1186/s12915-020-00775-7.
Braak, H., Rüb, U., Gai, W. P., & Del Tredici, K. (2003). Idiopathic Parkinson's disease. Possible routes by which vulnerable neuronal types may be subject to neuroinvasion by an unknown pathogen. Journal of neural transmission (Vienna, Austria: 1996), 110(5), 517-536.
Braak, H., De Vos, R. A. I., Bohl, J., & Del Tredici, K. (2006). Gastric α-synuclein immunoreactive inclusions in Meissner's and Auerbach's plexuses in cases staged for Parkinson's disease-related brain pathology. Neuroscience Letters, 396(1), 67-72.
Cairns, A. G., Vazquez-Romero, A., Mandi Moein, M., Ådén, J., Elmore, C S., Takano,

A., Arakawa, R., Varrone, A., Almqvist, F., & Schou, M. (2018). Increased Brain Exposure of an Alpha-Synuclein Fibrillization Modulator by Utilization of an Activated Ester Prodrug Strategy. Chem Neurosci., 9(11), 2542-2547.
Chaudhuri, K. R., & Schapira, A. H., (2009). Non-motor symptoms of Parkinson's disease: dopaminergic pathophysiology and treatment, Lancet Neurol. 8(5), 464-74.
Chermenina, M., Chorell, E., Pokrzywa, M., Antti, H., Almqvist, F., Strömberg, I. & Pernilla Wittung-Stafshede (2015). Single injection of small-molecule amyloid accelerator results in cell death of nigral dopamine neurons in mice. npj Parkinson's Disease, volume 1, Article number: 15024.
Dorsey, E. R., Sherer, T., Okun, M. S., & Bloemd, B. R. (2018). The emerging evidence of the Parkinson pandemic. Journal of Parkinson's Disease, 8(s1), S3-S8.
Fleming S M, Ekhator O R, Ghisays V. Assessment of sensorimotor function in mouse models of Parkinson's disease. J Vis Exp. 2013;(76):50303. Published 2013 Jun. 17. doi:10.3791/50303.
Glaab, E., Trezzi, J.-P., Greuel, A., Jäger, C., Hodak, Z., Drzezga, A., Timmermann, L., Tittgemeyer, M., Diederich, N. J., & Eggers, C. (2019) Integrative analysis of blood metabolomics and PET brain neuroimaging data for Parkinson's disease, Neurobiology of Disease. 124,555-562.
Guan Y, Han F. Key Mechanisms and Potential Targets of the NLRP3 Inflammasome in Neurodegenerative Diseases. Front Integr Neurosci. 2020;14:37. Published 2020 Jul. 24. doi:10.3389/fnint.2020.00037.
Harishankar, M. K., Sasikala, & C., Ramya, S. M. (2003). Efficiency of the intestinal bacteria in the degradation of the toxic pesticide, chlorpyrifos. 3 Biotech., 3(2): 137-142.
Horvath, I., Weise C. F., Andersson E. K., Chorell, E., Sellstedt, M., Bengtsson, C., Olofsson, A., Hultgre, S. J., Chapman, M., Wolf-Watz, M., Almqvist, F., & Wittung-Stafshede, P. (2012). Mechanisms of Protein Oligomerization: Inhibitor of Functional Amyloids Templates α-Synuclein Fibrillation, J. Am. Chem. Soc., 134, 7, 3439-3444.
Jung, P. P., Christian, N., Kay, D. P., Skupin, A., & Linster, C. L. (2015). Protocols and programs for high-throughput growth and aging phenotyping in yeast, PLoS One, 10(3).
Keshavarzian, A., Green, S. J., Engen, P. A., Voigt, R. M., Naqib, A., Forsyth, C. B., . . . Shannon, K. M. (2015). Colonic bacterial composition in Parkinson's disease. Movement Disorders, 30(10), 1351-1360.
Mollenhauer, B., Zimmermann, J., Sixel-Döring, F., Focke, N. K., Wicke, T., Ebentheuer, J., Schaumburg, M., Lang, E., Trautmann, E., Zetterberg, H., Taylor, P., Friede, T., & Trenkwalder, C. DeNoPa Study Group. (2016). Monitoring of 30 marker candidates in early Parkinson disease as progression markers. Neurology, 87(2):168-77.
Olanow, C. W., & Kieburtz K. (2010). Defining disease-modifying therapies for PD-A road map for moving forward. Mov Disord., 25(12):1774-9.
Scheperjans, F., Aho, V., Pereira, P. A. B., Koskinen, K., Paulin, L., Pekkonen, E., . . . Auvinen, P. (2015). Gut microbiota are related to Parkinson's disease and clinical phenotype. Movement Disorders, 30(3), 350-358.
Scheperjans, F., Pekkonen, E., Kaakkola, S., & Auvinen, P. (2015). Linking Smoking, Coffee, Urate, and Parkinson's Disease—A Role for Gut Microbiota?. J Parkinsons Dis., 5(2):255-62.
Schrag, A., Horsfall, L., Walters, K., Noyce, A., Petersen, I. (2015). Prediagnostic presentations of Parkinson's disease in primary care: a case-control study. Lancet Neurol., 14(1):57-64.
Simonsen, A. H., Kuiperij, B., El-Agnaf, O. M., Engelborghs, S., Herukka, S. K., Parnetti, L., Rektorova, I., Vanmechelen, E., Kapaki, E., Verbeek, M., & Mollenhauer, B. (2016). The utility of α-synuclein as biofluid marker in neurodegenerative diseases: a systematic review of the literature. Biomark Med., 10(1):19-34.
Vogt S., Lyon E. J., Shima S., and Thauer R. K. (2008). The exchange activities of [Fe] hydrogenase (iron-sulfur-cluster-free hydrogenase) from methanogenic archaea in comparison with the exchange activities of [FeFe] and [NiFe] hydrogenases. J Biol Inorg Chem, 13(1): 97.

1. An inhibitor of an iron-containing hydrogenase of a methanogenic archaeon for use in the treatment or prevention of a parkinsonian condition,
wherein said archaeon is in the gut of a subject and is characterized by the production of 2-hydroxypyridine.

2. Inhibitor for use according to claim 1, wherein the inhibitor prevents or reduces the production of 2-hydroxypyridine.

3. Inhibitor for use according to claim 2, wherein the inhibitor prevents or reduces the production of 2-hydroxypyridine by the methanogenic archaeon by blocking the synthesis pathway of 2-hydroxypyridine.

4. Inhibitor for use according to claim 2 or 3, wherein the inhibitor prevents or reduces the production of 2-hydroxypyridine by the methanogenic archaeon by targeting the methanogenic archaeon.

5. Inhibitor for use according to any one of the preceding claims, wherein the inhibitor prevents or reduces the production of 2-hydroxypyridine by preventing the degradation of a precursor of 2-hydroxypridine, preferably wherein the precursor of 2-hydroxypridine is derived from a pesticide, more preferably wherein the pesticide is chlorpyrifos.

6. Inhibitor for use according to any one of the preceding claims, wherein the inhibitor is selected from the group consisting of statins, preferably atorvastatin, fluvastatin, lovastatin, pitavastatin, pravastatin, rosuvastatin or simvastatin, and antibiotics, preferably rifaximin and neomycin.

7. Inhibitor for use according to any one of the preceding claims, wherein 2-hydroxypyridine comprises the compound of general formula I, 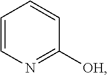
an isomer of said 2-hydroxypyridine or a mixture of isomers thereof;
a derivative of said 2-hydroxypyridine or a mixture of derivatives thereof;
a pharmaceutically acceptable salt of said 2-hydroxypyridine or a mixture of pharmaceutically acceptable salts thereof; or
a complex of said 2-hydroxypyridine or a mixture of complexes thereof.

8. Inhibitor for use according to any one of the preceding claims, wherein the inhibitor prevents or reduces the production of 2-hydroxypyridine in the gut of said subject.

9. Inhibitor for use according to any one of the preceding claims, wherein the inhibitor is a small molecule, preferably a small molecule having a molecular weight of less than 2000 daltons; or a biologic drug, preferably a peptide, a protein, an antibody or an antibody fragment.

10. Inhibitor for use according to any one of the preceding claims, wherein the inhibitor is for use in the treatment or prevention of a parkinsonian condition in co-treatment with other compounds, which are preferably selected from the group consisting of carbidopa-levodopa, dopamine agonists, MAO B inhibitors, catechol O-methyltransferase (COMT) inhibitors, anticholinergics, amantadine and antibiotics.

11. Inhibitor for use according to any one of the preceding claims, wherein the subject is administered a therapeutically effective amount of the inhibitor, preferably at least 0.1 mg.

12. Inhibitor for use according to any one of the preceding claims, wherein the subject is administered a dosage of the inhibitor in the range of 0.1 to 1.0 mg per day.

13. Inhibitor for use according to any one of the preceding claims, wherein the parkinsonian condition is selected from the group consisting of Parkinson's disease, Parkinsonism, Rapid Eye Movement Sleep Behavior Disorder and Parkinson-plus syndrome.

14. Kit comprising the inhibitor for use according to any one of the preceding claims.

15. The kit according to claim 14, further comprising a pharmaceutically acceptable carrier and/or an antiparkinsonian agent,
preferably said antiparkinsonian agent is one or more of the following: L-DOPA, deprenyl, apomorphine, an anticholinergic agent, further preferably said anticholinergic agent is selected from the group consisting of benzhexol and orphenadrine.

16. Pharmaceutical composition comprising the inhibitor for use according to any one of claims 1 to 13.

17. Method of screening for an inhibitor for use according to any one of claims 1 to 13, wherein the method comprises
(i) contacting a compound of interest with an iron-containing hydrogenase of a methanogenic archaeon,
(ii) measuring inhibitor function of the compound of interest for the iron-containing hydrogenase of the methanogenic archaeon,
wherein an increase of inhibition of said iron-containing hydrogenase compared to the iron-containing hydrogenase before contacting of step (i) indicates that the compound of interest serves as an inhibitor for the iron-containing hydrogenase, or
wherein a decrease or maintenance of inhibition of said iron-containing hydrogenase compared to the iron-containing hydrogenase before contacting of step (i) indicates that the compound of interest does not serve as an inhibitor for the iron-containing hydrogenase.

18. Method for determining whether or not a subject is susceptible to a treatment with an inhibitor for use according to any one of the claims 1 to 13, (i) comprising determining the concentration of 2-hydroxypyridine in a sample obtained from the gut of said subject, compared to a control sample of the same subject.
